SEQUENCE LISTING (25 sequences)

SEQ ID NO: 1 - DNA, 8366 bp, Foot-and-mouth disease virus
aagggggcactagggtctagccccagcgtcgcgtaacgaccgtccccggttgaaacgccactactcagacctctggctgtcgtacctccattaggcagacaggaaccacccttctggggcctacgctactgtgcccttttggggcccgcggtcgtcatcaccgcgcctccattaggctcgcagtcgtacctcctctaggctgacaactgtcgcccttttagggcctacgaccttcaagttacggttttggcgtccctcccgcggccgactcgcctacgtgaacgctagctcttcacccgaaggcccgcctttcaccccccccccccccccccccctaaagaacgtttccgtcttttccgacgttaaaggattgtaaccaaacgcttcttgtcgtcttttccgacgttaaaggattgtaaccacacgaattaccgtcttttccgacgttaaaggattgcaaccacacgatttgccttcttttccgaagtaaaaggatacaagcacacagttttgcccgttttcatgagaaatgggacgtctgcgcacgaaacgcgctgttgctcttcgcattcttgtacaaacacgatcttgacgcaggaatcttagaccaacccaacccgtgcacctgcaagtttcgcccggtccttccgggtcttgagagacaaacagatgtactgagatcaactccacgattggtctactagcgggtactagtaacactcactttgcttcgtagcggagcacatgagcggtgggacctcccccatggtaacatggacccaccgggccaaaagccacgcctcacggcctcatgtgtgtgcaaccccagcacggcaacttgtttgtgaaacacaccttaaggtaacactgagactggtacttgatttctggagacaggctaaggatgcccttcaggtaccccgaggtaacaagagacacttcgggatctgagaaggggaccaggagttctatcaaactgcccggtttaaaaagcttctatgcctggataggtgaccggaggccggcaccttttcctttatttaaactcactttatgaagacaactgactgtttcaacgttttgctcgagatcatttacaggttcaggcacacgtttaaaacagacaggaagatggaattcacactctacaacggagaaaagaagaccttctacagcaggcccaacaaacacgggaactgttggctcaactcacttctgcagctctttcgatacgtcgatgagccactctttgagtctgagtacctgtcacctgaaaacaaaacactggacatgatcaaacagctatctgattacaccaaattggacctgtcagacggagggccccccgctctcgttcttcggctgatcaaagattgtcttcagactggcgttggcaccagcactcgccccagcgagatctgtgtcatcaacggggttgtcatgaccctggctgatttccacgccggcattttcatcaaaggcactggacacgccgtgttcgccctcaacacatccgagggctggtatgccattgatgatgaggtgttctacccttggacacccgaccctgaaaacgtactcgcgtacgtcccctacgaccaggaaccactggacgtagactggcaagatcgcgcgggtctgttcctccgtggagcaggccactcatcacctgtcacagggtcacaaaaccaatctggcaatactggtagtatcatcaacaattactacatgcaacagtaccagaattcaatggacacccaacttggcgacaacgccatctcgggcgggtccaacgagggcagcactgacaccacgtctacccacacaaacaacacgcagaacaatgattggttttcaaaattggcccagtcagcgatctcggggcttttcggagccctcctcgcagacaaaaagacagaggaaaccactctgctcgaggaccgaatattgacaacacgacacggtacaaccacctccaccacacagagttccgttggcatcacctacggttacgctgacgctgactctttccgccccggacccaacacatcgggcctggagacgcgtgtggaacaagcagagcggttcttcaaggaaaagctttttgattggacatcagacaaaccatttggcacgctgtatgttttggaattgcccaaggaccacaaggggatctacggcagcctgaccgacgcgtatacttacatgcgcaacggttgggacgtccaggtttccgccaccagcacgcagttcaacggcgggtcactccttgtggccatggtgccggagctgtgctcgctcaaggacagagaggagtttcaactctctctctacccacaccagtttatcaacccaaggaccaacaccacagcacacatccaggtgccctacctcggtgtgaacaggcacgatcagggcaagcgccaccaggcgtggtccctggtcgtcatggtcctcacgcctctcaccaccgaggcacaaatgcaatccgggactgttgaggtttacgccaacatcgccccgacgaacgtcttcgttgctggcgaaaagcctgcgaaacagggcatcattccagttgcctgtttcgacggctatggtggattccaaaacaccgacccgaagaccgcagatcccatctacggttacgtgtacaacccgtctcgcaacgattgtcacggcaggtactccaacctgttggacgtcgccgaggcgtgccccactttcctgaactttgatggtaagccctacgtcgtcaccaagaacaacggcgacaaggtcatgacctgttttgatgtggcattcacgcacaaagttcacaagaacacgtttcttgcgggcctagcggattactacgcccagtaccagggttcgctgaactaccacttcatgtacacaggtcctactcaccataaagcaaagttcatggttgcctacatcccaccaggcattgagactgacagactgcccaagacacccgaggacgcagcccactgctaccactcggagtgggacacaggactgaactcccagttcacgttcgccgtcccatacgtctctgcaagtgacttctcctacacacacactgacacccccgcaatggcaaccaccaacggctgggtggcggtgttccaggtgactgacacccattcggccgaagccgctgtggttgtgtcggtgagcgctggacccgacctggagttcaggttcccggttgacccagtgcgccaaaccaccagctcaggtgaaggagcggacgtcgtgacgaccgacccttcgacccacggtggtgctgtcacagagaagaaacgtgtgcacacagacgtggcattcgtcatggacagattcacccatgttctgacaaatagaaccgcgttcgcggttgacttgatggacaccaacgagaagaccctggtaggcgcgctgctgcgtgcggccacctactatttctgtgacctggaaattgcctgccttggcgaacacgaacgcgtgtggtggcagccaaacggggcaccgcggacaaccacgcttcgcgacaaccccatggtgttttcacacaacaacgtcacgcgttttgctgtcccgtacaccgcgccacaccggctgctatcaaccagatacaacggtgagtgcaagtacacgcagcagtccactgccattcgcggtgaccgtgccgtcttggccgcaaagtacgccaacaccaaacacaaactcccgtctaccttcaacttcggctacgtgaccgccgacaaaccagtcgacgtttactaccggatgaagagggcggagctctactgtccaagacctctcctccctggctacgaccacgcagacagggacaggtttgacagccccattggtgttaagaaacaactgtgcaacttcgacctgttgaagttggctggagacgttgagtccaaccccgggcccttcttcttctccgacgtcagggagaacttcacaaagctggtggaaagcattaacaacatgcaacaagatatgtccactaaacacggacccgacttcaaccgtctggtgtccgcatttgaggaactgacaaaaggagtcaaggctatcaaggacggtctcgatgaggccaaaccctggtacaaagtcatcaaactcctcagccgcctgtcgtgcatggccgctgtcgcagctcgctccaaggatcccgtccttgtggcgatcatgctagctgacaccggtctcgagattctggacagcactttcgtcgtgaagaaaatctccgacgcgctctccagcgttttccacgtcccggcccctgtcttcagcttcggagccccgatcctgttggcaggtttggtcaaggtcgcctccacgttcttccggtccacacccgaagacttggagagagcagagaaacagctcaaggcacgtgacatcaacgacatcttcgccatcctcaagaacggcgaatggctggtcaaacttatcctggctatccgcgactggatcaaagcctggatttcctcagaggagaagtacatctccatgacggaccttgtgccgcgcattcttgaatgccagcacaacctgaacgatccgtccaagtaccaggaaagcaaagagtggctggaaaacgctcgcgaggcttgcctcaagaacggaaaccaccacattgccaacctgtgtaaagtgaatgcaccagcacccagcaggtcgagacccgaacccgtggtcgtttgtctccgtggcaaatccggccagggcaagagtttccttgctaacgtgctcgcacaagcaatctcaactcacttcactggcagaaccgactctgtctggtactgcccgcctgaccccgaccacttcgatggctataaccaacaaactgtcgttgttatggatgatttgggccagaaccctgacggcaaggacttcaagtacttcgcccagatggtctccactacagggttcatccctcccatggcttcactcgaagacaagggaaaaccgttcaacagcaaggtgatcattgcgacgagcaacctttattctgggttcacacccaggacaatggtctgccccgacgctttgaaccgacggttccactttgacattgatgtgagtgccaaggacgggtacaaagttaacaacagattggacatcatcaaagcactggaggacacgcacacaaacgcacccgccatgttcaattacgattgtgcccttctcaatggctccgccgttgaaatgaagagactgcaacaagatgtgttcaagcctctgccacctctcaacagcctgtaccagctggttgatgaagtgatagagagggtgaagctccacgagaaagtgtcgagccacccgattttcaagcaaatttccattccttcccaaaagtccgtgctctacttcctcatcgagaaaggacagcacgaagcagcaattgaattttacgagggaatggtgcacgacagcattaaggaagaacttaagcccttgttggagcaaaccagcttcgccaagcgtgcttttaaacgcctcaaggaaaacttcgagatcgttgctctcgttgttgtgctgttggcaaacatcatcatcatgatccgcgagactcgcaagcgccagaagatggtggacgatgctctcgatgagtacattgagaaggcaaacatcaccaccgacgacaaaacgcttgaagaggcgggaaggaaccctcaagaggttgtcgacaaacccactgtcggcttccgcgagagaaaactccctgggcataaaactgacgatgaagtgaactctgagccagccaaacccacggagaaaccacaagctgaaggaccctacgctggccccctcgagcgacagcagccgctaaagctcaaggccaagctccctcaggcagaggggccttacgccgggccgctagagaaacaacaaccactgaaactgaaagcgagactgcctgtggccaaggaagggccatatgaaggaccagtgaagaaacctgtcgctttgaaagtgaaagcaaaagccccgattgtcactgaaagcggatgcccaccgaccgacttgcaaaagatggtcatggcaaacgtgaagcccgttgagctcatcctcgacgggaagacagttgcgctctgctgcgcgactggagtgttcgggacggcttacctcgtgcctcgtcatcttttcgcagagaagtatgacaagatcatgctggacggccgcgccctgacagacagtgacttcagagtgtttgagttcgaggtgaaagtgaaaggacaggacatgctttcagatgccgcgctgatggttctccactctggaaaccgagtgcgtgatctcacggggcacttccgtgacaccatgaaactgtcgaaaggcagccccgtcgttggcgtggtcaacaacgccgacgtcggaagactcatcttctcaggagacgcactaacctacaaagacctagtcgtttgtatggacggtgacaccatgcctggactcttcgcgtaccgcgctgggaccaaaggttggatactgtggagccgctgttctcgcaaaggacggcgccaaaacagtgatcgtcggcacccactctgccggaggcaacggagtaggctactgctcctgcgtctcacgatccatgctcctgcagatgaaggcccacatcgaccctccccctcacactgaggggttggtagtggacaccagagaagttgaggagcgcgtgcacgtcatgcgcaaaaccaagctagcacccaccgtggctcacggtgtgtttcagcctgaatttggacctgccgccctgtcgaacaacgacaaacgcctgagcgaaggcgtggttttggacgaagtcatcttctccaaacacaagggtgatgccaaaatgtctgaggctgacaagagactgttccgcctgtgcgctgctgattatgcctcgcatcttcacaacgtacttgggacagccaactctccactgagcgtgtttgaagccatcaagggcgtcgacggacttgacgccatggagcctgacacagcacccggcctcccctgggcactccggggaaagcgccgcggagctctcatcgatttcgagaacggcactgtcggatccgagattgaagcggctctgaagctcatggagaagaaggagtacaagttcacctgtcaaaccttcctgaaggacgagattcgccctctggagaaagtcaaggccggcaagactcgcattgtcgacgtcttgcctgttgaacacatcatctataccagaatgatgattggcagattctgtgcacaaatgcactccaacaacggaccgcaaattggctcggcggtcggttgcaaccctgatgttgattggcaacgatttggcacccactttgcccagtacaaaaatgtttgggacattgactattcggcctttgatgctaatcattgcagtgacgccatgaacatcatgttcgaggaggtcttccgtgaggaatttggatttcatccgaacgctgtttggattctcaagactctcatcaacacggaacatgcctacgagaacaagcgcatcactgttgaaggcggaatgccctcgggctgctccgccaccagcatcatcaacaccattctcaacaacatctacgtgctttacgccctgcgtaggcactatgagggagtcgagctgtcgcactacaccatgatttcctacggggatgacattgtagttgcaagtgattacgatttggactttgaagctctcaagcctcactttaaatctcttggtcaaacgatcactccagccgacaaaagtgacaaaggttttgttcttggtcagtccatcaccgatgttactttcctcaagaggcattttcatctggattatgaaactgggttttacaaacctgtgatggcttcgaagaccctcgaagccatcctctcctttgcacgccgtgggaccatccaggagaagttgatctccgtggcaggactcgccgtccactccggacaagacgagtaccggcgtctcttcgaaccctttcagggaacgttcgagattccaagctacagatcactttacctgcgttgggtgaacgccgtgtgcggtgacgcataatccctcagagtacacaattggcagaaagtctctgaggcgagcgacgccgtaggagtggaaaggccgagaggccttttcccgcttccctaatccaaaaaaaaaaaaagcggccgccatggtcccagcctcctcgctggcgccggctgggcaacattccgaggggaccgtcccctcggtaatggcgaatgggacggggccgggctgctaacaaagcccgaaaggaagctgagttggctgctgccaccgctgagcaataactagcataaccccttggggcctctaaacgggtcttgaggggtttttt

SEQ ID NO: 2 - DNA, 8381 bp, Foot-and-mouth disease virus
aagggggcgctagggtctagccctagtgtcacgtaacgaccgtccccggttgaaacgctactactcagacctctggctgtcgtacctctattaggcaggcaggaaccacccttttggggcctacgcacaaccgcccttttggggcccgcggccgtcatcgtcgcgcctcctctaggctggtcatcgtacctcctctaggctgacaactgtcgcccttctagggcctacgaccctcaagttacggttttagcgtccctcccgcggccgactcgcctacgtgaacgctagctctccacccgaaggcctacctttcaccccccccccccccccccccctaaagaacgtttccgtcttttccgacgttaatggattgaaaccaaacgcttcttaccgtcttttccgacgtcaaaggattgtaaccatacgaactaccgtcgtttccgacgttaaaggattgtaaccacacgcaataccttcttttccgaagtaaaaggatacaaacacacagttttgcccgttttcatgagaaatgggacgtttgcgcacgaaacgcgccgttgctctttgcattcttgtacaaacacgatctcacgcaggaatcttagaccaacccaaaccgtgcaactgcaagtttcgcccggtctttccgggtctagagagacaaacagatgtactgagactgactccacgatcggtctactagcgggtgctagtaacactcattttgcttcgtagcggagcacgtgagcggtgggaactcccccatggtaacatggacccaccgggccaaaagccacgcctaacggcctcacgtgtgtgcaaccctagcacggcaacttgtttgtgaaacacatcttaaggtaacactgagactggtacttagtttctggagacaggctaaggatgcccttcaggtaccccgaggtaacaagagacacttcgggatctgagaaggggattgggagttctataaaactgcccagtttaaaaagcttctatgcctgaataggtgaccggaggccggcaccttttcctttttaccaaacaattttatgaagacaactgactgttttaacgttttgttcgagatcttccacagactccggcacacgttcaaagcagaaaggaaaatggaattcacactttacaacggtgaaaagaagaccttttacagcagacccaacgaacacggtaactgctggctcaactcactgttgcagctctttcgatacgtcgatgagccgctctttgagtcagagtatctgtcaccagagaacaagacactggacatgatcagacagctttctgattacaccaagcttgacctctccgacggtgggccacctgcactcgtgctctggctcatcaaggactgtcttcagaccggcgttggcaccagcactcgccccagcgagatctgtgtgatcaacggggttgtcatgaccctagctgattttcacgccggaattttcatcaaaggtaccgaacacgctgtgttcgctctcaacacatctgagggctggtacgccattgatgatgaggtgttctacccatggacaccggaccctgagaacgtactcgcgtacgtgccctacgaccaagaaccattggacgttgactggcaggaccgcgctggtctgttcctccgcggtgcgggccagtcgtcacccgccacagggtcacaaaatcaatcaggtaacacaggtagtatcatcaacaactactacatgcaacaataccaaaattcaatggacacacaactcggagacaacgccatctctggcgggtcaaacgaggggtcgaccgacacgacgtcgacccacaccaacaacacccagaacaatgattggttttcaaaattggcacaatcggccttttccggcctggttggtgctctgcttgctgacaagaagaccgaggaaaccactcttctcgaagatcgtatcctcaccaccagccacggcacaaccacctcaaccacgcaaagttcagttggcgtaacctacggatatgccgagtctgaccactttctacccggcccaaacactaacgggctggagacacgcgtggaacaggccgagaggttcttcaaacacaaactctttgattggacacttgaacaacaatttggaacaacccacattctggagctgcccacagaccacaagggtatttatgggcaactggtcgactcccactcttacatccgtaacgggtgggatgttgaggtctccgcgaccgcaacccagtttaatggtggttgcctcttggtagccatggtgcccgagctgtgcaaactggctgatcgagagaagtaccaactaactctcttccctcaccagttcctgaacccaaggaccaacaccacggcacacatccaggtaccctatctaggagtggaccgacacgatcaggggacacgccacaaggcgtggactttggttgtcatggtggtggcgccttacaccaacgaccagacaattggctcgaccaaagccgaggtgtacgtgaacatagcacccacaaatgtttacgttgccggtgagaaacccgcaaaacagggcattctccccgtggctgtttctgacggttacggcggcttccaaaacacagatcccaaaacatcggatcccgtttacgggcacgtgtacaacccagcccgtactggcctgcctgggaggttcacaaacctcctggacgtggccgaagcgtgtcccacgtttcttgacttcaacggtgtgccgtatgtcaccacccagtccaattctgggtctaaagtgctaacacgttttgatttggcttttgggcacaaaaatttgaaaaacactttcatgtctggtcttgcccagtactacgcgcagtacagtggcacactcaacctgcatttcatgtacacaggcccaacaaacaacaaggcaaagtacatggtggcctacatccctcccgggacacaccctctcccggaaactccggagatggcgtcccactgttaccacgctgaatgggacacaggcctgaattcaaccttcaccttcaccgtgccgtacgtatcggccgctgactacgcgtacacctactctgacgaacctgaacaggcttcggtccagggttgggtgggtgtgtaccaggtgacctacacacacgagaaagacggtgcagttgtcgtatctatcagtgctggacccgacttcgagttcaggatgcccatcatcccttcccgccagacaacatctgctggggaaggcgcggagcccgtcacagttgacgcctcccaacacggtggcaacagccgcggtgtccacaggcaacacactgatgtcagtttcctgcttgaccggttcacgctggttggcaagacacagaacaacaaaatgacacttgatctactccagaccaaagaaaaagcactggttggcgcaatcctgcgtgcgggcacgtactacttctctgacttggaggtggcgtgtctcggtgaaaacaaatgggtcggctggactcctaacggagcaccagaacttgaggaagttggcgacaacccagtcgtcttttccaaacgaggagccacccgctttgcattgccgttcactgccccacacaggtgtcttgcaacaacttacaatggtgattgcaagtacaaacccgctggcacggccccgcgcgacaacatccgcggggacctcgcagtccttgcacagaggattgccggcgagacacacattccaaccactttcaactacggcaggatttacactgaggccgaagtggacgtgtacgtcaggatgaaacgcgcggaactctactgcccgcgtcctctcttgacacactacgaccacaatggcaaggatcgctacaagacagcgataaccaaacctgccaaacaacttgggaactttgaactgttgaagttggccggagacgttgagtccaaccctgggcccttcttcttcgctgacgtcagggaaaacttcaccaagttggtggacagcattaacagcatgcaacaagacatgtccactaaacacggacccgacttcaaccgtctggtgtccgcatttgaggaactgacacaaggagttaaagccatcaaggaagggctcgacgaggccaagccttggtacaaagtcatcaaactcctcagccgcctgtcgtgcatggccgctgtcgcagctcgctccaaggatcccgtcctagtcgcgatcatgctagctgacaccggtctcgagattctggacagcactttcgtcgtgaagaaaatctccgacgcgctctccagcgttttccacgtcccggcccctgtcttcagtttcggtgccccgattctgttggcaggtttggtcaaggtcgcctccacgttcttccggtccacacccgaagacctggagagagcagaaaagcagctcaaggcacgtgacatcaacgacatcttcgccatcctcaagaacggcgaatggctggtcaaacttatcctggcaatccgcgactggatcaaagcttggatttcctcagaggagaaatacatctccatgacggatcttgtgccgcgcattctcgagtgccagcgcaacctcaacgatccgtccaagtaccaggagagcaaagagtggctcgaaaacgctcgcgaggcctgcctcaagaacggaaacgtccacattgctaacctgtgtaaagtgaatgcaccggcacccagcaagtcgagacccgagccggtggtcgtttgcctccgcggcaaatccggccaaggcaagagtttccttgcgaacgtgcttgcacaagcaatctcaacccacttcactggacgtgtggactcagtctggtactgtccacctgaccctgaccacttcgacggctacaaccaacaggccgttgttgtgatggatgatttgggccagaaccctgacggcaaggacttcaagtacttcgcccagatggtctctaccacagggttcatccctcccatggcttcgctcgaggacaagggaaaaccgttcaacagcaaggtcctcattgcgacgagcaacctttactctgggttcacgcctagggcgatggtctgccccgacgctctgaaccgacggtttcactttgacatcgacgtgagtgccaaggacgggtacaaagttaacaacagattggacatcatcaaagcactggaggatacgcacacaaacgcgcccgccatgttcaactatgactgtgcccttctcaatggctccgccgttgaaatgaagagactgcaacaagatgtgttcaagcctctgccacctctcaacagcctgtaccaactggttgatgaagtgatagagagggtgaagctccacgagaaagtgtcgagccacccgattttcaagcagatttccattccttcccaaaagtctgtgctttacttcctcattgagaaaggacagcacgaagctgcaattgaattctacgaagggatggtgcacgacagcatcaaggaagagcttaaaccgcttctcgagcaaaccagcttcgcgaagcgtgccttcaagcgcctgaaggaaaacttcgagatcgttgctctcgtcgttgtgctgttggcaaacattgttatcatgatccgcgaaactcgcaagagacagaagatggtcgatgacgccctcgatgaatacattgagaaggcgaacatcaccactgatgacaaaactcttgacgaggcggaaaggaaccctcaggaggttgtcgacaaacccactgtcggcttccgtgagaggagactccccgggcacaagactgacgatgaagtgaacactgagccagtcaagcccgcggagagaccacaagctgaaggaccctacgcgggaccgctcgaacgacagtagccgctgaagctcaaggccaaactgcccagagcagagggcccttacgcgggaccgctagagaaacaacaaccactgaaactgaaagccagattgcctgtggccaaagaagggccatatgaaggaccagtcaagaagcctgtcgcttggaaagtgaaagcaaaagccccgattgtcactgaaagcggatgcccaccgaccgacttgcaaaagatggttatggcaaacgtgaagcccgttgagctcatccgcgacgggaagaccgttgcgctctgttgcgctacgggagtgttcgggacggcctacctcgtgcctcgtcaccttttcgcagagaagtatgacaagatcatgttggacggccgtgccctgacagactgtgacttcagagtgtttgagtttgaggtaaaagtaaaaggacaggacatgctctcagatgccgcgctcatggttctccactctggaaaccgcgtgcgcgatctcacgggacacttccgtgacatcatgaaactgtcgaaaggcagtcccgtcgttggtgttgtcaacaacgctgacgtcggaagactcatcttctcaggagatgcactgacttacaaagacctggtcgtttgtatggacggtgacaccatgcctggactcttcgcctaccgcgcagggaccaaggttggatactgcggagctgctgtcctcgcaaaggatggcgccaagactgtgatcgtcggcacccactcggccggaggtaacggagtaggctactgctcctgcgtctctcgatccatgctcctgcagatgaaggcccacatcgacccaccccttcacaccgaggggctggtagtagacaccagagaagttgaggagcgcgtgcatgtcatgcgcaaaaccaagcttgcacacaccgtagcttacggtgtgtttcagcctgaatttggacctgccgccctgtcaaacaacgacaagcgcctgaacgaaggcgtggtcttggacgaagtcatcttctccaagcacaagggcgatgccaaaatgtctgaggctgataagaaactgttccgcctgtgcgctgctgattatgcctcgcatcttcacaacgtgcttgggacagcaaactctccactgagcgtgtttgaagccatcaagggcgtcgacggactcgacgccatggagcctgacacagcacccggtcttccctgggccctccaggggcaacgccgcggagctctcatcgatttcgagaacggcactgtcggacccgagattgaacaggcactgaagctcatggagaagaaggagtacaagttcacctgccaaaccttcctgaaggacgagattcgcccactggagaaagtcaaggccggcaagactcgcattgtcgacgtcctgcccgtggaacacatcatctacaccagaatgatgattggcagattttgtgcgcaaatgcactccaacaacggaccgcaaattggctcggcggtcggttgcaaccctgatgttgattggcaaagattcggctgtcatttcgcccagtacagaaatgtttgggacattgactattcggcctttgatgctaaccattgcagtgacgccatgaacatcatgttcgaggaggttttccgtgaagaatttggatttcatccgaacgctgtttggattctcaaaactctcatcaacacggaacacgcctacgagaacaagcgcatcactgttgaaggtggaatgccctcgggttgctccgccaccagcatcatcaacaccattctcaacaacatctacgtgctctacgccctgcgtagacactatgagggagtcgagctgtcgcactacaccatgatttcctacggggatgatattgtggtcgcaagtgattacgatttggactttgaagctctcaagcctcacttcaaatctcttggtcaaacaatcactccagccgacaaaagtgacaaaggttttgttcttggtcagtccattaccgatgttactttcctcaagaggcacttccatctggattatggaactgggttttacaaacctgtgatggcttcgaagaccctcgaagctatcctctcctttgcacgccgtgggaccatccaggagaagttgatctccgtggcaggactcgccgtccactccggacctgacgagtaccggcgtctcttcgaaccctttcagggaacgttcgagattccaagctacagatcactttacctgcgttgggtgaacgccgtgtgcggtgacgcataatccctcagaacacacaattggcagaacgtgtctgaggcgagcgacgccgtaggagtgaaaaggccgaaaggccttttcccgcttccctaacccaaaaaaaaaaaaaaagaagcggccgccatggtcccagcctcctcgctggcgccggctgggcaacattccgaggggaccgtcccctcggtaatggcgaatgggacggggccgggctgctaacaaagcccgaaaggaagctgagttggctgctgccaccgctgagcaataactagcataaccccttggggcctctaaacgggtcttgaggggtttttt

SEQ ID NO: 3 - protein, 644 aa, Artificial Sequence
  Outer capsid protein of chimeric Foot and Mouth Disease Virus serotype SAT1/KPN/196/91
TTRHGTTTSTTQSSVGITYGYADSDRFLPGPNTNGLETRVEQAERFFKHKLFDWTLEQRFGTTHVLELPTDHKGIYGQLVDSHSYIRNGWDVEVSATATQFNGGCLLVAMVPELCKLSEREKYQLTLFPHQFLNPRTNTTAHIQVPYLGVDRHDQGTRHKAWTLVVMVVAPYTNDQTIGSNKAEVYVNIAPTNVYVAGEKPAKQGILPVAVSVGYGGFQNTDPKTSDPVYGHVYNPARTGLPGRFTNLLDVAEACPTLLDFNGVPYVTTQANSGSKVLTCFDLAFGHKNLKNTFMSGLAQYYTQYSGTLNLHFMYTGPTNNKAKYMVAYIPPGTHPLPETPEMASHCYHAEWDTGLNSTFTFTVPYVSAADFAYTYSDEPEQASVQGWVGVYQVTDTHEKDGAVVVSVSAGPDFEFRMPISPSRQTTSAGEGAEPVTTDASQHGGDRRTTRRHHTDVSFLLDRFTLVGKTQDNKLTLDLLQTKEKALVGAILRAATYYFSDLEVACVGDNKWVGWTPNGAPELAEVGDNPVVFSKGRTTRFALPYTAPHRCLATAYNGDCKYKPTGTAPRENIRGDLATLAARIASETHIPTTFNYGRIYTDTEVDVYVRMKRAELYCPRPVLTHYDHGGRDRYRTAITKPVKQ

SEQ ID NO: 4 - protein, 644 aa, Artificial Sequence
  Outer capsid protein of chimeric Foot and Mouth Disease Virus serotype SAT1/NAM/307/98
TTRHGTTTSTTQSSVGVTYGYSLTDKFLPGPNTNGLETRVEQAERFFKHKLFDWTLEQQFGTTYVMELPTDHKGIYGQLVDSHAYIRNGWDVQVSATATQFNGGCLLVAMVPELCKLGEREKYQLTLFPHQFLNPRTNTTAHIQVPYLGVDRHDQGTRHKAWTLVVMVLAPYTNDQTIGSTKAEVYVNISPTNVYVAGEKPSKQGIFPVAVSDGYGGFQNTDPKTSDPIYGHVYNPARTLYPGRFTNLLDVAEACPTLLDFNGVPYVQTQNNSGSKVLTCFDLAFGHKNMKNTYMSGLAQYFAQYSGTLNLHFMYTGPTNNKAKYMVAYIPPGTNPLPETPEMASHCYHAEWDTGLNSTFTFTVPYISAADYAYTYADEPEQASVQGWVGVYQITDTHEKDGAVVVSVSAGPDFEFRMPISPSRQTTSAGEGAEPVTTDASAHGGSARTTRRAHTDVAFLLDRFTLVGKTKDNKLVLDLLSTKEKTLVGALLRAATYYFSDLEVACVGTNAWVGWTPNGSPVLTEVGDNPVVFSRGGTTRFALPYTAPHRVLATVYNGDCKYKPTGTPPRENIRGDLATLAKRIASETHIPTTFNYGMIYTEAEVDVYLRMKRAELYCPRPVLTHYDHGGKDRYKTALVRPAKQ

SEQ ID NO: 5 - protein, 641 aa, Artificial Sequence
  Outer capsid protein of chimeric Foot and Mouth Disease Virus serotype SAT2/SAU/6/00
VTTRHGTTTSTTQSSVGVTLGYADADSFRPGPNTSGLETRVQQAERFFKEKLFDWTSDKPFGTLYVLELPKDHKGIYGKLTDSYTYMRNGWDVQVSATSTQFNGGSLLVAMVPELSSLKSREEFQLTLYPHQFINPRTNTTAHIQVPYLGVNRHDQGKRHHAWSLVVMVLTPLTTEAQMNSGTVEVYANIAPTNVVVAGELPGKQGIVPVAAADGYGGFQNTDPKTADPIYGYVYNPSRNDCHGRFSNLLDVAEACPTLLDFDGKPYIVTKNNGDKVMTSFDVAFTHKVHRNTFLAGLADYYTQYSGSLNYHFMYTGPTHHKAKFMVAYVPPGVETAQLPTTPEDAAHCYHAEWDTGLNSSFSFAVPYISAADFSYTHTDTPAMATTNGWVIVLQVTDTHSAEAAVVVSVSAGPDLEFRFPIDPVRQTTSAGESADVVTTDPSTHGGNVQEGRRKHTEVAFLLDRSTHVHTNKTSFVVDLMDTKEKALVGAILRASTYYFCDLEIACVGDHTRAFWQPNGAPRTTQLGDNPMVFAKGGVTRFAIPFTAPHRLLSTVYNGECVYKKTPTAIRGDRAALAVKYADSTHTLPSTFNFGFVTVDKPVDVYYRMKRAELYCPRPLLPAYEHTGGDRFDAPIGVERQ

SEQ ID NO: 6 - protein, 99 aa, Foot-and-mouth disease virus
  misc_feature (1)..(99): pSAT2_1 without its native capside protein
GAGHSSPVTGSQNQSGNTGSIINNYYMQQYQNSMDTQLGDNAISGGSNEGSTDTTSTHTNNTQNNDWFSKLAQSAISGLFGALLADKKTEETTLLEDRI

SEQ ID NO: 7 - protein, 98 aa, Foot-and-mouth disease virus
  misc_feature (1)..(98): pSAT2_2 without its native capsid protein
AGHSSPVTGSQNQSGNTGSIINNYYMQQYQNSMDTQLGDNAISGGSNEGSTDTTSTHTNNTQNNDWFSKLAQSAISGLFGALLADKKTEETTLLEDRI

SEQ ID NO: 8 - protein, 744 aa, Foot-and-mouth disease virus
  misc_feature (1)..(744): SAR/09/81 Impala Epith
GAGQSSPATGSQNQSGNTGSIINNYYMQQYQNSMDTQLGDNAISGGSNEGSTDTTSTHTNNTQNNDWFSKLAQSAFSGLVGALLADKKTEETTLLEDRILTTSHGTTTSTTQSSVGVTYGYAESDHFLPGPNTNGLETRVEQAERFFKHKLFDWTLEQQFGTTHILELPTDHKGIYGQLVDSHSYIRNGWDVEVSATATQFNGGCLLVAMVPELCKLADREKYQLTLFPHQFLNPRTNTTAHIQVPYLGVDRHDQGTRHKAWTLVVMVVAPYTNDQTIGSTKAEVYVNIAPTNVYVAGEKPAKQGILPVAVSDGYGGFQNTDPKTSDPVYGHVYNPARTGLPGRFTNLLDVAEACPTFLDFNGVPYVTTQSNSGSKVLTRFDLAFGHKNLKNTFMSGLAQYYAQYSGTLNLHFMYTGPTNNKAKYMVAYIPPGTHPLPETPEMASHCYHAEWDTGLNSTFTFTVPYVSAADYAYTYSDEPEQASVQGWVGVYQVTDTHEKDGAVVVSISAGPDFEFRMPISPSRQTTSAGEGAEPVTVDASQHGGNSRGVHRQHTDVSFLLDRFTLVGKTQNNKMTLDLLQTKEKALVGAILRAATYYFSDLEVACLGENKWVGWTPNGAPELEEVGDNPVVFSNRGATRFALPFTAPHRCLATTYNGDCKYKPAGTAPRDNIRGDLAVLAQRIAGETHIPTTFNYGRIYTEAEVDVYVRMKRAELYCPRPLLTHYDHNGKDRYKTAITKPAKQ

SEQ ID NO: 9 - protein, 744 aa, Foot-and-mouth disease virus
  misc_feature (1)..(744): KNP/196/91 PK1
GAGQSSPATGSQNQSGNTGSIINNYYMQQYQNSMDTQLGDNAISGGSNEGSTDTTSTHTNNTQNNDWFSKLAQSAFSGLVGALLADKKTEETTLLEDRILTTSHGTTTSTTQSSVGITYGYADSDRFLPGPNTNGLETRVEQAERFFKHKLFDWTLEQQFGTTHVLELPTDHKGIYGQLVDSHSYIRNGWDVEVSATATQFNGGCLLVAMVPELCKLSEREKYQLTLFPHQFLNPRTNTTAHIQVPYLGVDRHDQGTRHKAWTLVVMVVAPYTNDQTIGSSKAEVYVNIAPTNVYVAGEKPAKQGILPVAVSDGYGGFQNTDPKTSDPVYGHVYNPARTGLPGRFTNLLDVAEACPTLLDFNGVPYVTTQANSGSKVLTCFDLAFGHKNLKNTFMSGLAQYYTQYSGTLNLHFMYTGPTNNKAKYMVAYIPPGTHPLPETPEMASHCYHAEWDTGLNSTFTFTVPYVSAADFAYTYSDEPEQASVQGWVGVYQVTDTHEKDGAVVVSVSAGPDFEFRMPISPSRQTTSAGEGAEPVTTDASQYGGDRRTTRRHHTDVSFLLDRFTLVGKTQDNRLTLDLLQTKEKALVGAILRAATYYFSDLEVACVGDNKWVGWTPNGAPELAEVGDNPVVFSKGGTTRFALPYTAPHRCLATAYNGDCKYKPTGTAPRENIRGDLATLAARIASETHIPTTFNYGRIYTDTVVDVYVRMKRAELYCPRPVLTHYDHGGKDRYKTAITKPVKQ

SEQ ID NO: 10 - protein, 744 aa, Foot-and-mouth disease virus
  misc_feature (1)..(744): NAM/307/98/1 PK1RS4
GAGQSSPATGSQNQSGNTGSIINNYYMQQYQNSMDTQLGDNAISGGSNEGSTDTTSTHTNNTQNNDWFSKLAQSAFSGLVGALLADKKTEETTLLEDRIMTTSHGTTTSTTQSSVGVTYGYSLTDKFLPGPNTNGLETRVEQAERFFKHKLFDWTLEQQFGTTYVMELPTDHKGIYGQLVDSHAYIRNGWDVQVSATATQFNGGCLLVAMVPELCKLGEREKYQLTLFPHQFLNPRTNTTAHIQVPYLGVDRHDQGTRHKAWTLVVMVLAPYTNDQTIGSTKAEVYVNISPTNVYVAGEKPSKQGIFPVAVSDGYGGFQNTDPKTSDPIYGHVYNPARTLYPGRFTNLLDVAEACPTLLDFNGVPYVQTQNNSGSKVLTCFDLAFGHKNMKNTYMSGLAQYFAQYSGTLNLHFMYTGPTNNKAKYMVAYIPPGTNPLPETPEMASHCYHAEWDTGLNSTFTFTVPYISAADYAYTYADEPEQASVQGWVGVYQITDTHEKDGAVVVSVSAGPDFEFRMPISPSRQTTSAGEGAEPVTTDASAHGGSARTTRRAHTDVAFLLDRFTLVGKTKDNKLVLDLLSTKEKTLVGALLRAATYYFSDLEVACVGTNAWVGWTPNGSPVLTEVGDNPVVFSRGGTTRFALPYTAPHRVLATVYNGDCKYKPTGTPPRENIRGDLATLAKRIASETHIPTTFNYGMIYTEAEVDVYLRMKRAELYCPRPVLTHYDHGGKDRYKTALVRPAKQ

SEQ ID NO: 11 - protein, 744 aa, Foot-and-mouth disease virus
  misc_feature (1)..(744): ZAM/2/93 PK1RS3
GAGQSSPATGSQNQSGDTGSIINNYYMQQYQNSMDTQLGDNAISGGSNEGSTDTTSTHTNNTQNNDWFSKLAQSAFSGLVGALLADKKTEETTLLEDRILTTSHGTTTSTTQSSVGVTCGYADTDRFLPGPNTNGLETRVKQAERFFKHKLFDWTSEQKFGTTHVLELPTDHKGIYGQLVDSHSYIRNGWDVEVSATATQFNGGCLLVAMVPELCKLSDREKYQLTLFPHQFLNPRTNTTAHIQVPYLGVDRHDQGTRHKTWTLVVMVVAPYTNDQTIGSTKAEVYVNIAPTNVYVAGEKPAKHGIIPVAVADGYGGFQNTDPKTSDPIYGHVYNPARTALPGRFTNLLDVAEACPTLLDFNGVPYVTTRNNSGDKILACFDLAFGHKNLKNTYMSGLAQYYTQYSGTLNIHFMYTGPTNNKAKYMVAYIPPGTHPLPTTPEMASHCYHAEWDTGLNSTFTFTVPYISAADFAYTYSDEPEQASAQGWVAVYQITDTHEKDGAVVVTVSAGPDFEFRMPISPSRQTTSAGEGAEPVTTDASQHGGNRRATRRQHTDVSFLLDRFTLVGKTVENKLTLDLLQTKEKALVGAILRAATYYFSDLEVACVGTNKWVGWTPNGAPELSEVGDNPVVFSHNGTTRFALPYTAPHRCLATTYNGNCKYKPATEAPPTHVRGDLATLAARIASETHIPTTFNYGRIYTEAEVDVYVRMKRAELYCPRPVLTHYDHQGKDRYKVALTKPAKQ

SEQ ID NO: 12 - protein, 740 aa, Foot-and-mouth disease virus
  misc_feature (1)..(740): KNP/19/89 PK1RS2
GAGQSSPATGSQNQSGNTGSIINNYYMQQYQNSMDTQLGDNAISGGSNEGSTDTTSTHTNNTQNNDWFSKLAQSAISGLFGALLADKKTEETTLLEDRIVTTRHGTTTSTTQSSVGITYGYADADSFRPGPNTSGLETRVQQAERFFKEKLFDWTPEKPFGTLYVLELPKDHKGIYGSLTEAYTYMRNGWDVQVTATSTQFNGGSLLVAMVPELCSLRDREEFQLSLYPHQFINPRTNTTAHIQVPYLGVNRHDQGKRHQAWSLVVMVLTPLTTETQMNSGTVEVYANIAPTNVFVAGEKPAKQGIIPVACSAGYGGFQNTDPKTADPIYGYVYNPSRNHCHGRYSTLLDVAQACPTFLNFDGKPYVVTKNNGDKVMTCFDVAFTHKVHKNTFLAGLADYYTQYQGSLNYHFMYTGPTHHKAKFMVAYIPPGIETEKLPKTPEDAAHCYHSEWDTGLNSQFTFAVPYVSASDFSYTHTDTPAMATTNGWVAVYQVPDTHSAEAAVVVSVSAGPDLEFRFPIDPVRQTTSAGEGADVVTTDPSTHGGQVVEKRRMHTDVAFVLDRFTHVHTNKTTFNVDLMDTKDKTLVGALLRASTYYFCDLEIACVGDHQRVYWQPNGAPRTRELGDNPMVFSNKGVTRFAVPYTAPHRLLSTVYNGECKYETPVTAIRGDRAVLAAKYSNIKHTLPSTFNFGHVTADNSVDVYYRMKRAELYCPRPLFPAYDYASRDRFHVPIGVEKQ

SEQ ID NO: 13 - protein, 740 aa, Foot-and-mouth disease virus
  misc_feature (1)..(740): ZIM/07/83/2
GAGHSSPVTGSQNQSGNTGSIINNYYMQQYQNSMDTQLGDNAISGGSNEGSTDTTSTHTNNTQNNDWFSKLAQSAISGLFGALLADKKTEETTLLEDRIVTTRHGTTTSTTQSSVGITYGYADADSFRPGPNTSGLETRVEQAERFFKEKLFDWTSDKPFGTLYVLELPKDHKGIYGSLTDAYTYMRNGWDVQVSATSTQFNGGSLLVAMVPELCSLKDREEFQLSLYPHQFINPRTNTTAHIQVPYLGVNRHDQGKRHQAWSLVVMVLTPLTTEAQMQSGTVEVYANIAPTNVFVAGEKPAKQGIIPVACFDGYGGFQNTDPKTADPIYGYVYNPSRNDCHGRYSNLLDVAEACPTFLNFDGKPYVVTKNNGDKVMTCFDVAFTHKVHKNTFLAGLADYYAQYQGSLNYHFMYTGPTHHKAKFMVAYIPPGIETDRLPKTPEDAAHCYHSEWDTGLNSQFTFAVPYVSASDFSYTHTDTPAMATTNGWVAVFQVTDTHSAEAAVVVSVSAGPDLEFRFPVDPVRQTTSSGEGADVVTTDPSTHGGAVTEKKRVHTDVAFVMDRFTHVLTNRTAFAVDLMDTNEKTLVGGLLRAATYYFCDLEIACLGEHERVWWQPNGAPRTTTLRDNPMVFSHNNVTRFAVPYTAPHRLLSTRYNGECKYTQQSTAIRGDRAVLAAKYANTKHKLPSTFNFGHVTADKPVDVYYRMKRAAVYCPRPLLPGYDHADRDRFDSPIGVEKQ

SEQ ID NO: 14 - protein, 740 aa, Foot-and-mouth disease virus
  misc_feature (1)..(740): ZIM/05/83 BTY4RS1
GAGHSSPATGSQNQSGNTGSIINNYYMQQYQNSMDTQLGDNAISGGSNEGSTDTTSTHTNNTQNNDWFSKLAQSAISGLFGALLADKKTEETTLLEDRIVTTRHGTTTSTTQSSVGITYGYADADSFRPGPNTSGLETRVEQAERFFKEKLFDWTSDKPFGMLYVLELPKDHKGIYGSLTDAYTYMRNGWDVQVSATSTQFNGGSLLVAMVPELCSLKDREEFQLSLYPHQFINPRTNTTAHIQVPYLGVNRHDQGKRHQAWSLVVMVLTPLTTEAQMQSGTVEVYANIAPTNVFVAGEKPAKQGIIPVACFDGYGGFQNTDPKTADPIYGYVYNPSRNDCHGRYSNLLDVAEACPTFLNFDGKPYVFTKNNGDKVMTCFDVAFTHKVHKNTFLAGLADYYAQYQGSLNYHFMYTGPTHHKAKFMVAYIPPGIETDRLPKTPEDAAHCYHSEWDTGLNSQFTFAVPYVSASDFSYTHTDTPAMATTNGWVAVFQVTDTHSAEAAVVVSVSAGPDLEFRFPVDPVRQTTSSGEGADVVTTDPSTHGGAVTEKKRMHTDVAFVMDRFTHVLTNRTAFAVDLMDTNEKTLVGALLRAATYYFCDLEIACLGEHERVWWQPNGAPRTTTLRDNPMVFSHNNVTRFAVPYTAPHRLLSTRYNGECKYTQQSTAIRGDRAVLAAKYANTKHELPSTFNFGYVTADKPVDVYYRMKRAELYCPRPLLPGYDHADRDRFDSPIGVEKQ

SEQ ID NO: 15 - protein, 740 aa, Foot-and-mouth disease virus
  misc_feature (1)..(740): ZIM/14/90/2 BTY1RS3
GAGQSSPATGSQNQSGNTGSIINNYYMQQYQNSMDTQLGDNAISGGSNEGSTDTTSTHTNNTQNNDWFSKLAQSAISGLFGALLADKKTEETTLLEDRIVTTRHGTTTSTTQSSVGITYGYADSDSFRSGPNTSGLETRVEQAERFFKEKLFDWTSDKPFGTLYILELPKDHKGIYGSLTESYAYMRNGWDVQVSATSTQFNGGSLLVAMVPELCSLRAREEFQLSLYPHQFINPRTNTTAHIQVPYLGVNRHDQGKRHQAWSLVVMVLTPLTTEAQMNSGTVEVYANIAPTNVFVAGEMPAKQGIIPVACSDGYGGFQNTDPKTADPIYGYVYNPSRNDCHGRYSNLLDVAEACPTFLDFDGKPYVVTKNNGDKVMTCFDVAFTHKVHKSTFLAGLADYYTQYQGSLNYHFMYTGPTHHKAKFMVAYIPPGTATDKLPKTPEDAAHCYHSEWDTGLNSQFTFAVPYVSASDFSYTHTDTPAMATTNGWVAVYQVTDTHSAEAAVVVSVSAGPDLEFRFPIDPIRQTTSSGEGADVVTTDPSTHGGSVAEKRRMHTDVAFVMDRFTHVHTNKTAFAVDLMDTNEKTLVGALLRASTYYFCDLEIACIGDHKRVWWQPNGAPRTTQLRDNPMVFSHNSVTRFALPYTAPHRLLSTRYNGECNYTQRSPAIRGDRAVLAAKYANVKHELPSTFNFGFVTADKPVDVYFRMKRTELYCPRPLLPAYDHGDRDRFDAPIGVEKQ

SEQ ID NO: 16 - protein, 740 aa, Foot-and-mouth disease virus
  MISC_FEATURE (1)..(740): ZAM/7/96 BTY1RS2
GAGQSSPATGSQNQSGNTGSIINNYYMQQYQNSMDTQLGDNAISGGSNEGSTDTTSTHTNNTQNNDWFSKLAQSAISGLFGALLADKKTEETTLLEDRIVTTRHGTTTSTTQSSVGITYGYADADSFRPGPNTSGLETRVEQAERFFKEKLFDWTSDKPFGTLYVLELPKDHKGIYGSLTDAYAYMRNGWDVQVTATSTQFNGGSLLVALVPELCSLREREEFQLTLYPHQFINPRTNTTAHIQVPYLGVNRHDQGKRHQAWSLVVMVLTPLTTETQMTSGTVEVYANIAPTNVFVAGEMPAKQGIVPVACADGYGGFQNTDPKTADPIYGYVYNPSRNDCHGRYSNLLDVAEACPTLLNFDGKPYVVTKNNGDKVMTCFDVAFTHKVHKNTFLAGLADYYTQYQGSLNYHFMYTGPTHHKAKFMVAYIPPGVETDKLPKTPEDAAHCYHSEWDTGLNSQFTFAVPYVSASDFSYTHTDTPAMATTNGWVAVYQVTDTHSAEAAVVVSVSAGPDLEFRFPIDPVRQTTSAGEGADVVTTDPSTHGGRVVEKRRMHTDVAFVLDRFTHVHTNKTTFNVDLMDTKEKTLVGALLRASTYYFCDLEIACVGEHARVYWQPNGAPRTTQLGDNPMVFSHNKVTRFAIPYTAPHRLLATRYNGECKYTQEARAIRGDRAVLAAKYAGAKHSLPSTFNFGHVTADAAVDVYYRMKRAELYCPRPLLPAYEHSDRDRFDAPIGVEKQ

SEQ ID NO: 17 - protein, 744 aa, Artificial Sequence
  pSAT2_1 and SAT1/KPN/196/91
GAGHSSPVTGSQNQSGNTGSIINNYYMQQYQNSMDTQLGDNAISGGSNEGSTDTTSTHTNNTQNNDWFSKLAQSAISGLFGALLADKKTEETTLLEDRILTTRHGTTTSTTQSSVGITYGYADSDRFLPGPNTNGLETRVEQAERFFKHKLFDWTLEQRFGTTHVLELPTDHKGIYGQLVDSHSYIRNGWDVEVSATATQFNGGCLLVAMVPELCKLSEREKYQLTLFPHQFLNPRTNTTAHIQVPYLGVDRHDQGTRHKAWTLVVMVVAPYTNDQTIGSNKAEVYVNIAPTNVYVAGEKPAKQGILPVAVSVGYGGFQNTDPKTSDPVYGHVYNPARTGLPGRFTNLLDVAEACPTLLDFNGVPYVTTQANSGSKVLTCFDLAFGHKNLKNTFMSGLAQYYTQYSGTLNLHFMYTGPTNNKAKYMVAYIPPGTHPLPETPEMASHCYHAEWDTGLNSTFTFTVPYVSAADFAYTYSDEPEQASVQGWVGVYQVTDTHEKDGAVVVSVSAGPDFEFRMPISPSRQTTSAGEGAEPVTTDASQHGGDRRTTRRHHTDVSFLLDRFTLVGKTQDNKLTLDLLQTKEKALVGAILRAATYYFSDLEVACVGDNKWVGWTPNGAPELAEVGDNPVVFSKGRTTRFALPYTAPHRCLATAYNGDCKYKPTGTAPRENIRGDLATLAARIASETHIPTTFNYGRIYTDTEVDVYVRMKRAELYCPRPVLTHYDHGGRDRYRTAITKPVKQ

SEQ ID NO: 18 - protein, 743 aa, Artificial Sequence
  pSAT2_2 and SAT1/NAM/307/98
AGHSSPVTGSQNQSGNTGSIINNYYMQQYQNSMDTQLGDNAISGGSNEGSTDTTSTHTNNTQNNDWFSKLAQSAISGLFGALLADKKTEETTLLEDRIVTTRHGTTTSTTQSSVGVTYGYSLTDKFLPGPNTNGLETRVEQAERFFKHKLFDWTLEQQFGTTYVMELPTDHKGIYGQLVDSHAYIRNGWDVQVSATATQFNGGCLLVAMVPELCKLGEREKYQLTLFPHQFLNPRTNTTAHIQVPYLGVDRHDQGTRHKAWTLVVMVLAPYTNDQTIGSTKAEVYVNISPTNVYVAGEKPSKQGIFPVAVSDGYGGFQNTDPKTSDPIYGHVYNPARTLYPGRFTNLLDVAEACPTLLDFNGVPYVQTQNNSGSKVLTCFDLAFGHKNMKNTYMSGLAQYFAQYSGTLNLHFMYTGPTNNKAKYMVAYIPPGTNPLPETPEMASHCYHAEWDTGLNSTFTFTVPYISAADYAYTYADEPEQASVQGWVGVYQITDTHEKDGAVVVSVSAGPDFEFRMPISPSRQTTSAGEGAEPVTTDASAHGGSARTTRRAHTDVAFLLDRFTLVGKTKDNKLVLDLLSTKEKTLVGALLRAATYYFSDLEVACVGTNAWVGWTPNGSPVLTEVGDNPVVFSRGGTTRFALPYTAPHRVLATVYNGDCKYKPTGTPPRENIRGDLATLAKRIASETHIPTTFNYGMIYTEAEVDVYLRMKRAELYCPRPVLTHYDHGGKDRYKTALVRPAKQ

SEQ ID NO: 19 - protein, 739 aa, Artificial Sequence
  pSAT2_2 and SAT2/SAU/6/00
AGHSSPVTGSQNQSGNTGSIINNYYMQQYQNSMDTQLGDNAISGGSNEGSTDTTSTHTNNTQNNDWFSKLAQSAISGLFGALLADKKTEETTLLEDRIVTTRHGTTTSTTQSSVGVTLGYADADSFRPGPNTSGLETRVQQAERFFKEKLFDWTSDKPFGTLYVLELPKDHKGIYGKLTDSYTYMRNGWDVQVSATSTQFNGGSLLVAMVPELSSLKSREEFQLTLYPHQFINPRTNTTAHIQVPYLGVNRHDQGKRHHAWSLVVMVLTPLTTEAQMNSGTVEVYANIAPTNVVVAGELPGKQGIVPVAAADGYGGFQNTDPKTADPIYGYVYNPSRNDCHGRFSNLLDVAEACPTLLDFDGKPYIVTKNNGDKVMTSFDVAFTHKVHRNTFLAGLADYYTQYSGSLNYHFMYTGPTHHKAKFMVAYVPPGVETAQLPTTPEDAAHCYHAEWDTGLNSSFSFAVPYISAADFSYTHTDTPAMATTNGWVIVLQVTDTHSAEAAVVVSVSAGPDLEFRFPIDPVRQTTSAGESADVVTTDPSTHGGNVQEGRRKHTEVAFLLDRSTHVHTNKTSFVVDLMDTKEKALVGAILRASTYYFCDLEIACVGDHTRAFWQPNGAPRTTQLGDNPMVFAKGGVTRFAIPFTAPHRLLSTVYNGECVYKKTPTAIRGDRAALAVKYADSTHTLPSTFNFGFVTVDKPVDVYYRMKRAELYCPRPLLPAYEHTGGDRFDAPIGVERQ

SEQ ID NO: 20 - DNA, 10831 bp, Artificial Sequence
  pGEM cloning vector containing wild type Foot and Mouth Disease Virus serotype SAT2 genome length cDNA, with hammerhead and hepatitis delta virus ribozymes at the 5′ and 3′ ends of the genome, respectively
ttgaaagggggcactagggtctagccccagcgtcgcgtaacgaccgtccccggttgaaacgccactactcagacctctggctgtcgtacctccattaggcagacaggaaccacccttctggggcctacgctactgtgcccttttggggcccgcggtcgtcatcaccgcgcctccattaggctcgcagtcgtacctcctctaggctgacaactgtcgcccttttagggcctacgaccttcaagttacggttttggcgtccctcccgcggccgactcgcctacgtgaacgctagctcttcacccgaaggcccgcctttcaccccccccccccccccccccctaaagaacgtttccgtcttttccgacgttaaaggattgtaaccaaacgcttcttgtcgtcttttccgacgttaaaggattgtaaccacacgaattaccgtcttttccgacgttaaaggattgcaaccacacgatttgccttcttttccgaagtaaaaggatacaagcacacagttttgcccgttttcatgagaaatgggacgtctgcgcacgaaacgcgctgttgctcttcgcattcttgtacaaacacgatcttgacgcaggaatcttagaccaacccaacccgtgcacctgcaagtttcgcccggtccttccgggtcttgagagacaaacagatgtactgagatcaactccacgattggtctactagcgggtactagtaacactcactttgcttcgtagcggagcacatgagcggtgggacctcccccatggtaacatggacccaccgggccaaaagccacgcctcacggcctcatgtgtgtgcaaccccagcacggcaacttgtttgtgaaacacaccttaaggtaacactgagactggtacttgatttctggagacaggctaaggatgcccttcaggtaccccgaggtaacaagagacacttcgggatctgagaaggggaccaggagttctatcaaactgcccggtttaaaaagcttctatgcctggataggtgaccggaggccggcaccttttcctttatttaaactcactttatgaagacaactgactgtttcaacgttttgctcgagatcatttacaggttcaggcacacgtttaaaacagacaggaagatggaattcacactctacaacggagaaaagaagaccttctacagcaggcccaacaaacacgggaactgttggctcaactcacttctgcagctctttcgatacgtcgatgagccactctttgagtctgagtacctgtcacctgaaaacaaaacactggacatgatcaaacagctatctgattacaccaaattggacctgtcagacggagggccccccgctctcgttcttcggctgatcaaagattgtcttcagactggcgttggcaccagcactcgccccagcgagatctgtgtcatcaacggggttgtcatgaccctggctgatttccacgccggcattttcatcaaaggcactggacacgccgtgttcgccctcaacacatccgagggctggtatgccattgatgatgaggtgttctacccttggacacccgaccctgaaaacgtactcgcgtacgtcccctacgaccaggaaccactggacgtagactggcaagatcgcgcgggtctgttcctccgtggagcaggccactcatcacctgtcacagggtcacaaaaccaatctggcaatactggtagtatcatcaacaattactacatgcaacagtaccagaattcaatggacacccaacttggcgacaacgccatctcgggcgggtccaacgagggcagcactgacaccacgtctacccacacaaacaacacgcagaacaatgattggttttcaaaattggcccagtcagcgatctcggggcttttcggagccctcctcgcagacaaaaagacagaggaaaccactctgctcgaggaccgaatattgacaacacgacacggtacaaccacctccaccacacagagttccgttggcatcacctacggttacgctgacgctgactctttccgccccggacccaacacatcgggcctggagacgcgtgtggaacaagcagagcggttcttcaaggaaaagctttttgattggacatcagacaaaccatttggcacgctgtatgttttggaattgcccaaggaccacaaggggatctacggcagcctgaccgacgcgtatacttacatgcgcaacggttgggacgtccaggtttccgccaccagcacgcagttcaacggcgggtcactccttgtggccatggtgccggagctgtgctcgctcaaggacagagaggagtttcaactctctctctacccacaccagtttatcaacccaaggaccaacaccacagcacacatccaggtgccctacctcggtgtgaacaggcacgatcagggcaagcgccaccaggcgtggtccctggtcgtcatggtcctcacgcctctcaccaccgaggcacaaatgcaatccgggactgttgaggtttacgccaacatcgccccgacgaacgtcttcgttgctggcgaaaagcctgcgaaacagggcatcattccagttgcctgtttcgacggctatggtggattccaaaacaccgacccgaagaccgcagatcccatctacggttacgtgtacaacccgtctcgcaacgattgtcacggcaggtactccaacctgttggacgtcgccgaggcgtgccccactttcctgaactttgatggtaagccctacgtcgtcaccaagaacaacggcgacaaggtcatgacctgttttgatgtggcattcacgcacaaagttcacaagaacacgtttcttgcgggcctagcggattactacgcccagtaccagggttcgctgaactaccacttcatgtacacaggtcctactcaccataaagcaaagttcatggttgcctacatcccaccaggcattgagactgacagactgcccaagacacccgaggacgcagcccactgctaccactcggagtgggacacaggactgaactcccagttcacgttcgccgtcccatacgtctctgcaagtgacttctcctacacacacactgacacccccgcaatggcaaccaccaacggctgggtggcggtgttccaggtgactgacacccattcggccgaagccgctgtggttgtgtcggtgagcgctggacccgacctggagttcaggttcccggttgacccagtgcgccaaaccaccagctcaggtgaaggagcggacgtcgtgacgaccgacccttcgacccacggtggtgctgtcacagagaagaaacgtgtgcacacagacgtggcattcgtcatggacagattcacccatgttctgacaaatagaaccgcgttcgcggttgacttgatggacaccaacgagaagaccctggtaggcgcgctgctgcgtgcggccacctactatttctgtgacctggaaattgcctgccttggcgaacacgaacgcgtgtggtggcagccaaacggggcaccgcggacaaccacgcttcgcgacaaccccatggtgttttcacacaacaacgtcacgcgttttgctgtcccgtacaccgcgccacaccggctgctatcaaccagatacaacggtgagtgcaagtacacgcagcagtccactgccattcgcggtgaccgtgccgtcttggccgcaaagtacgccaacaccaaacacaaactcccgtctaccttcaacttcggctacgtgaccgccgacaaaccagtcgacgtttactaccggatgaagagggcggagctctactgtccaagacctctcctccctggctacgaccacgcagacagggacaggtttgacagccccattggtgttaagaaacaactgtgcaacttcgacctgttgaagttggctggagacgttgagtccaaccccgggcccttcttcttctccgacgtcagggagaacttcacaaagctggtggaaagcattaacaacatgcaacaagatatgtccactaaacacggacccgacttcaaccgtctggtgtccgcatttgaggaactgacaaaaggagtcaaggctatcaaggacggtctcgatgaggccaaaccctggtacaaagtcatcaaactcctcagccgcctgtcgtgcatggccgctgtcgcagctcgctccaaggatcccgtccttgtggcgatcatgctagctgacaccggtctcgagattctggacagcactttcgtcgtgaagaaaatctccgacgcgctctccagcgttttccacgtcccggcccctgtcttcagcttcggagccccgatcctgttggcaggtttggtcaaggtcgcctccacgttcttccggtccacacccgaagacttggagagagcagagaaacagctcaaggcacgtgacatcaacgacatcttcgccatcctcaagaacggcgaatggctggtcaaacttatcctggctatccgcgactggatcaaagcctggatttcctcagaggagaagtacatctccatgacggaccttgtgccgcgcattcttgaatgccagcacaacctgaacgatccgtccaagtaccaggaaagcaaagagtggctggaaaacgctcgcgaggcttgcctcaagaacggaaaccaccacattgccaacctgtgtaaagtgaatgcaccagcacccagcaggtcgagacccgaacccgtggtcgtttgtctccgtggcaaatccggccagggcaagagtttccttgctaacgtgctcgcacaagcaatctcaactcacttcactggcagaaccgactctgtctggtactgcccgcctgaccccgaccacttcgatggctataaccaacaaactgtcgttgttatggatgatttgggccagaaccctgacggcaaggacttcaagtacttcgcccagatggtctccactacagggttcatccctcccatggcttcactcgaagacaagggaaaaccgttcaacagcaaggtgatcattgcgacgagcaacctttattctgggttcacacccaggacaatggtctgccccgacgctttgaaccgacggttccactttgacattgatgtgagtgccaaggacgggtacaaagttaacaacagattggacatcatcaaagcactggaggacacgcacacaaacgcacccgccatgttcaattacgattgtgcccttctcaatggctccgccgttgaaatgaagagactgcaacaagatgtgttcaagcctctgccacctctcaacagcctgtaccagctggttgatgaagtgatagagagggtgaagctccacgagaaagtgtcgagccacccgattttcaagcaaatttccattccttcccaaaagtccgtgctctacttcctcatcgagaaaggacagcacgaagcagcaattgaattttacgagggaatggtgcacgacagcattaaggaagaacttaagcccttgttggagcaaaccagcttcgccaagcgtgcttttaaacgcctcaaggaaaacttcgagatcgttgctctcgttgttgtgctgttggcaaacatcatcatcatgatccgcgagactcgcaagcgccagaagatggtggacgatgctctcgatgagtacattgagaaggcaaacatcaccaccgacgacaaaacgcttgaagaggcgggaaggaaccctcaagaggttgtcgacaaacccactgtcggcttccgcgagagaaaactccctgggcataaaactgacgatgaagtgaactctgagccagccaaacccacggagaaaccacaagctgaaggaccctacgctggccccctcgagcgacagcagccgctaaagctcaaggccaagctccctcaggcagaggggccttacgccgggccgctagagaaacaacaaccactgaaactgaaagcgagactgcctgtggccaaggaagggccatatgaaggaccagtgaagaaacctgtcgctttgaaagtgaaagcaaaagccccgattgtcactgaaagcggatgcccaccgaccgacttgcaaaagatggtcatggcaaacgtgaagcccgttgagctcatcctcgacgggaagacagttgcgctctgctgcgcgactggagtgttcgggacggcttacctcgtgcctcgtcatcttttcgcagagaagtatgacaagatcatgctggacggccgcgccctgacagacagtgacttcagagtgtttgagttcgaggtgaaagtgaaaggacaggacatgctttcagatgccgcgctgatggttctccactctggaaaccgagtgcgtgatctcacggggcacttccgtgacaccatgaaactgtcgaaaggcagccccgtcgttggcgtggtcaacaacgccgacgtcggaagactcatcttctcaggagacgcactaacctacaaagacctagtcgtttgtatggacggtgacaccatgcctggactcttcgcgtaccgcgctgggaccaaaggttggatactgtggagccgctgttctcgcaaaggacggcgccaaaacagtgatcgtcggcacccactctgccggaggcaacggagtaggctactgctcctgcgtctcacgatccatgctcctgcagatgaaggcccacatcgaccctccccctcacactgaggggttggtagtggacaccagagaagttgaggagcgcgtgcacgtcatgcgcaaaaccaagctagcacccaccgtggctcacggtgtgtttcagcctgaatttggacctgccgccctgtcgaacaacgacaaacgcctgagcgaaggcgtggttttggacgaagtcatcttctccaaacacaagggtgatgccaaaatgtctgaggctgacaagagactgttccgcctgtgcgctgctgattatgcctcgcatcttcacaacgtacttgggacagccaactctccactgagcgtgtttgaagccatcaagggcgtcgacggacttgacgccatggagcctgacacagcacccggcctcccctgggcactccggggaaagcgccgcggagctctcatcgatttcgagaacggcactgtcggatccgagattgaagcggctctgaagctcatggagaagaaggagtacaagttcacctgtcaaaccttcctgaaggacgagattcgccctctggagaaagtcaaggccggcaagactcgcattgtcgacgtcttgcctgttgaacacatcatctataccagaatgatgattggcagattctgtgcacaaatgcactccaacaacggaccgcaaattggctcggcggtcggttgcaaccctgatgttgattggcaacgatttggcacccactttgcccagtacaaaaatgtttgggacattgactattcggcctttgatgctaatcattgcagtgacgccatgaacatcatgttcgaggaggtcttccgtgaggaatttggatttcatccgaacgctgtttggattctcaagactctcatcaacacggaacatgcctacgagaacaagcgcatcactgttgaaggcggaatgccctcgggctgctccgccaccagcatcatcaacaccattctcaacaacatctacgtgctttacgccctgcgtaggcactatgagggagtcgagctgtcgcactacaccatgatttcctacggggatgacattgtagttgcaagtgattacgatttggactttgaagctctcaagcctcactttaaatctcttggtcaaacgatcactccagccgacaaaagtgacaaaggttttgttcttggtcagtccatcaccgatgttactttcctcaagaggcattttcatctggattatgaaactgggttttacaaacctgtgatggcttcgaagaccctcgaagccatcctctcctttgcacgccgtgggaccatccaggagaagttgatctccgtggcaggactcgccgtccactccggacaagacgagtaccggcgtctcttcgaaccctttcagggaacgttcgagattccaagctacagatcactttacctgcgttgggtgaacgccgtgtgcggtgacgcataatccctcagagtacacaattggcagaaagtctctgaggcgagcgacgccgtaggagtggaaaggccgagaggccttttcccgcttccctaatccaaaaaaaaaaaaagcggccgccatggtcccagcctcctcgctggcgccggctgggcaacattccgaggggaccgtcccctcggtaatggcgaatgggacggggccgggctgctaacaaagcccgaaaggaagctgagttggctgctgccaccgctgagcaataactagcataaccccttggggcctctaaacgggtcttgaggggttttttgctgaaaggaggaactatatccggaggcctatttaaatggccgcaattccggtctccctatagtgagtcgtattaatttcgataagccattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacaacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacaagctgactctagcatgctaatacgactcactatagggcctttcaactgatgaggccgaaaggccgaaaacccggtatcccgggtcc

SEQ ID NO: 21 - DNA, 10846 bp, Artificial Sequence
  pGEM cloning vector containing wild type Foot and Mouth Disease Virus serotype SAT1 genome length cDNA, with hammerhead and hepatitis delta virus ribozymes at the 5′ and 3′ ends of the genome, respectively
ttgaaagggggcgctagggtctagccctagtgtcacgtaacgaccgtccccggttgaaacgctactactcagacctctggctgtcgtacctctattaggcaggcaggaaccacccttttggggcctacgcacaaccgcccttttggggcccgcggccgtcatcgtcgcgcctcctctaggctggtcatcgtacctcctctaggctgacaactgtcgcccttctagggcctacgaccctcaagttacggttttagcgtccctcccgcggccgactcgcctacgtgaacgctagctctccacccgaaggcctacctttcaccccccccccccccccccccctaaagaacgtttccgtcttttccgacgttaatggattgaaaccaaacgcttcttaccgtcttttccgacgtcaaaggattgtaaccatacgaactaccgtcgtttccgacgttaaaggattgtaaccacacgcaataccttcttttccgaagtaaaaggatacaaacacacagttttgcccgttttcatgagaaatgggacgtttgcgcacgaaacgcgccgttgctctttgcattcttgtacaaacacgatctcacgcaggaatcttagaccaacccaaaccgtgcaactgcaagtttcgcccggtctttccgggtctagagagacaaacagatgtactgagactgactccacgatcggtctactagcgggtgctagtaacactcattttgcttcgtagcggagcacgtgagcggtgggaactcccccatggtaacatggacccaccgggccaaaagccacgcctaacggcctcacgtgtgtgcaaccctagcacggcaacttgtttgtgaaacacatcttaaggtaacactgagactggtacttagtttctggagacaggctaaggatgcccttcaggtaccccgaggtaacaagagacacttcgggatctgagaaggggattgggagttctataaaactgcccagtttaaaaagcttctatgcctgaataggtgaccggaggccggcaccttttcctttttaccaaacaattttatgaagacaactgactgttttaacgttttgttcgagatcttccacagactccggcacacgttcaaagcagaaaggaaaatggaattcacactttacaacggtgaaaagaagaccttttacagcagacccaacgaacacggtaactgctggctcaactcactgttgcagctctttcgatacgtcgatgagccgctctttgagtcagagtatctgtcaccagagaacaagacactggacatgatcagacagctttctgattacaccaagcttgacctctccgacggtgggccacctgcactcgtgctctggctcatcaaggactgtcttcagaccggcgttggcaccagcactcgccccagcgagatctgtgtgatcaacggggttgtcatgaccctagctgattttcacgccggaattttcatcaaaggtaccgaacacgctgtgttcgctctcaacacatctgagggctggtacgccattgatgatgaggtgttctacccatggacaccggaccctgagaacgtactcgcgtacgtgccctacgaccaagaaccattggacgttgactggcaggaccgcgctggtctgttcctccgcggtgcgggccagtcgtcacccgccacagggtcacaaaatcaatcaggtaacacaggtagtatcatcaacaactactacatgcaacaataccaaaattcaatggacacacaactcggagacaacgccatctctggcgggtcaaacgaggggtcgaccgacacgacgtcgacccacaccaacaacacccagaacaatgattggttttcaaaattggcacaatcggccttttccggcctggttggtgctctgcttgctgacaagaagaccgaggaaaccactcttctcgaagatcgtatcctcaccaccagccacggcacaaccacctcaaccacgcaaagttcagttggcgtaacctacggatatgccgagtctgaccactttctacccggcccaaacactaacgggctggagacacgcgtggaacaggccgagaggttcttcaaacacaaactctttgattggacacttgaacaacaatttggaacaacccacattctggagctgcccacagaccacaagggtatttatgggcaactggtcgactcccactcttacatccgtaacgggtgggatgttgaggtctccgcgaccgcaacccagtttaatggtggttgcctcttggtagccatggtgcccgagctgtgcaaactggctgatcgagagaagtaccaactaactctcttccctcaccagttcctgaacccaaggaccaacaccacggcacacatccaggtaccctatctaggagtggaccgacacgatcaggggacacgccacaaggcgtggactttggttgtcatggtggtggcgccttacaccaacgaccagacaattggctcgaccaaagccgaggtgtacgtgaacatagcacccacaaatgtttacgttgccggtgagaaacccgcaaaacagggcattctccccgtggctgtttctgacggttacggcggcttccaaaacacagatcccaaaacatcggatcccgtttacgggcacgtgtacaacccagcccgtactggcctgcctgggaggttcacaaacctcctggacgtggccgaagcgtgtcccacgtttcttgacttcaacggtgtgccgtatgtcaccacccagtccaattctgggtctaaagtgctaacacgttttgatttggcttttgggcacaaaaatttgaaaaacactttcatgtctggtcttgcccagtactacgcgcagtacagtggcacactcaacctgcatttcatgtacacaggcccaacaaacaacaaggcaaagtacatggtggcctacatccctcccgggacacaccctctcccggaaactccggagatggcgtcccactgttaccacgctgaatgggacacaggcctgaattcaaccttcaccttcaccgtgccgtacgtatcggccgctgactacgcgtacacctactctgacgaacctgaacaggcttcggtccagggttgggtgggtgtgtaccaggtgacctacacacacgagaaagacggtgcagttgtcgtatctatcagtgctggacccgacttcgagttcaggatgcccatcatcccttcccgccagacaacatctgctggggaaggcgcggagcccgtcacagttgacgcctcccaacacggtggcaacagccgcggtgtccacaggcaacacactgatgtcagtttcctgcttgaccggttcacgctggttggcaagacacagaacaacaaaatgacacttgatctactccagaccaaagaaaaagcactggttggcgcaatcctgcgtgcgggcacgtactacttctctgacttggaggtggcgtgtctcggtgaaaacaaatgggtcggctggactcctaacggagcaccagaacttgaggaagttggcgacaacccagtcgtcttttccaaacgaggagccacccgctttgcattgccgttcactgccccacacaggtgtcttgcaacaacttacaatggtgattgcaagtacaaacccgctggcacggccccgcgcgacaacatccgcggggacctcgcagtccttgcacagaggattgccggcgagacacacattccaaccactttcaactacggcaggatttacactgaggccgaagtggacgtgtacgtcaggatgaaacgcgcggaactctactgcccgcgtcctctcttgacacactacgaccacaatggcaaggatcgctacaagacagcgataaccaaacctgccaaacaacttgggaactttgaactgttgaagttggccggagacgttgagtccaaccctgggcccttcttcttcgctgacgtcagggaaaacttcaccaagttggtggacagcattaacagcatgcaacaagacatgtccactaaacacggacccgacttcaaccgtctggtgtccgcatttgaggaactgacacaaggagttaaagccatcaaggaagggctcgacgaggccaagccttggtacaaagtcatcaaactcctcagccgcctgtcgtgcatggccgctgtcgcagctcgctccaaggatcccgtcctagtcgcgatcatgctagctgacaccggtctcgagattctggacagcactttcgtcgtgaagaaaatctccgacgcgctctccagcgttttccacgtcccggcccctgtcttcagtttcggtgccccgattctgttggcaggtttggtcaaggtcgcctccacgttcttccggtccacacccgaagacctggagagagcagaaaagcagctcaaggcacgtgacatcaacgacatcttcgccatcctcaagaacggcgaatggctggtcaaacttatcctggcaatccgcgactggatcaaagcttggatttcctcagaggagaaatacatctccatgacggatcttgtgccgcgcattctcgagtgccagcgcaacctcaacgatccgtccaagtaccaggagagcaaagagtggctcgaaaacgctcgcgaggcctgcctcaagaacggaaacgtccacattgctaacctgtgtaaagtgaatgcaccggcacccagcaagtcgagacccgagccggtggtcgtttgcctccgcggcaaatccggccaaggcaagagtttccttgcgaacgtgcttgcacaagcaatctcaacccacttcactggacgtgtggactcagtctggtactgtccacctgaccctgaccacttcgacggctacaaccaacaggccgttgttgtgatggatgatttgggccagaaccctgacggcaaggacttcaagtacttcgcccagatggtctctaccacagggttcatccctcccatggcttcgctcgaggacaagggaaaaccgttcaacagcaaggtcctcattgcgacgagcaacctttactctgggttcacgcctagggcgatggtctgccccgacgctctgaaccgacggtttcactttgacatcgacgtgagtgccaaggacgggtacaaagttaacaacagattggacatcatcaaagcactggaggatacgcacacaaacgcgcccgccatgttcaactatgactgtgcccttctcaatggctccgccgttgaaatgaagagactgcaacaagatgtgttcaagcctctgccacctctcaacagcctgtaccaactggttgatgaagtgatagagagggtgaagctccacgagaaagtgtcgagccacccgattttcaagcagatttccattccttcccaaaagtctgtgctttacttcctcattgagaaaggacagcacgaagctgcaattgaattctacgaagggatggtgcacgacagcatcaaggaagagcttaaaccgcttctcgagcaaaccagcttcgcgaagcgtgccttcaagcgcctgaaggaaaacttcgagatcgttgctctcgtcgttgtgctgttggcaaacattgttatcatgatccgcgaaactcgcaagagacagaagatggtcgatgacgccctcgatgaatacattgagaaggcgaacatcaccactgatgacaaaactcttgacgaggcggaaaggaaccctcaggaggttgtcgacaaacccactgtcggcttccgtgagaggagactccccgggcacaagactgacgatgaagtgaacactgagccagtcaagcccgcggagagaccacaagctgaaggaccctacgcgggaccgctcgaacgacagtagccgctgaagctcaaggccaaactgcccagagcagagggcccttacgcgggaccgctagagaaacaacaaccactgaaactgaaagccagattgcctgtggccaaagaagggccatatgaaggaccagtcaagaagcctgtcgcttggaaagtgaaagcaaaagccccgattgtcactgaaagcggatgcccaccgaccgacttgcaaaagatggttatggcaaacgtgaagcccgttgagctcatccgcgacgggaagaccgttgcgctctgttgcgctacgggagtgttcgggacggcctacctcgtgcctcgtcaccttttcgcagagaagtatgacaagatcatgttggacggccgtgccctgacagactgtgacttcagagtgtttgagtttgaggtaaaagtaaaaggacaggacatgctctcagatgccgcgctcatggttctccactctggaaaccgcgtgcgcgatctcacgggacacttccgtgacatcatgaaactgtcgaaaggcagtcccgtcgttggtgttgtcaacaacgctgacgtcggaagactcatcttctcaggagatgcactgacttacaaagacctggtcgtttgtatggacggtgacaccatgcctggactcttcgcctaccgcgcagggaccaaggttggatactgcggagctgctgtcctcgcaaaggatggcgccaagactgtgatcgtcggcacccactcggccggaggtaacggagtaggctactgctcctgcgtctctcgatccatgctcctgcagatgaaggcccacatcgacccaccccttcacaccgaggggctggtagtagacaccagagaagttgaggagcgcgtgcatgtcatgcgcaaaaccaagcttgcacacaccgtagcttacggtgtgtttcagcctgaatttggacctgccgccctgtcaaacaacgacaagcgcctgaacgaaggcgtggtcttggacgaagtcatcttctccaagcacaagggcgatgccaaaatgtctgaggctgataagaaactgttccgcctgtgcgctgctgattatgcctcgcatcttcacaacgtgcttgggacagcaaactctccactgagcgtgtttgaagccatcaagggcgtcgacggactcgacgccatggagcctgacacagcacccggtcttccctgggccctccaggggcaacgccgcggagctctcatcgatttcgagaacggcactgtcggacccgagattgaacaggcactgaagctcatggagaagaaggagtacaagttcacctgccaaaccttcctgaaggacgagattcgcccactggagaaagtcaaggccggcaagactcgcattgtcgacgtcctgcccgtggaacacatcatctacaccagaatgatgattggcagattttgtgcgcaaatgcactccaacaacggaccgcaaattggctcggcggtcggttgcaaccctgatgttgattggcaaagattcggctgtcatttcgcccagtacagaaatgtttgggacattgactattcggcctttgatgctaaccattgcagtgacgccatgaacatcatgttcgaggaggttttccgtgaagaatttggatttcatccgaacgctgtttggattctcaaaactctcatcaacacggaacacgcctacgagaacaagcgcatcactgttgaaggtggaatgccctcgggttgctccgccaccagcatcatcaacaccattctcaacaacatctacgtgctctacgccctgcgtagacactatgagggagtcgagctgtcgcactacaccatgatttcctacggggatgatattgtggtcgcaagtgattacgatttggactttgaagctctcaagcctcacttcaaatctcttggtcaaacaatcactccagccgacaaaagtgacaaaggttttgttcttggtcagtccattaccgatgttactttcctcaagaggcacttccatctggattatggaactgggttttacaaacctgtgatggcttcgaagaccctcgaagctatcctctcctttgcacgccgtgggaccatccaggagaagttgatctccgtggcaggactcgccgtccactccggacctgacgagtaccggcgtctcttcgaaccctttcagggaacgttcgagattccaagctacagatcactttacctgcgttgggtgaacgccgtgtgcggtgacgcataatccctcagaacacacaattggcagaacgtgtctgaggcgagcgacgccgtaggagtgaaaaggccgaaaggccttttcccgcttccctaacccaaaaaaaaaaaaaaagaagcggccgccatggtcccagcctcctcgctggcgccggctgggcaacattccgaggggaccgtcccctcggtaatggcgaatgggacggggccgggctgctaacaaagcccgaaaggaagctgagttggctgctgccaccgctgagcaataactagcataaccccttggggcctctaaacgggtcttgaggggttttttgctgaaaggaggaactatatccggaggcctatttaaatggccgcaattccggtctccctatagtgagtcgtattaatttcgataagccattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacaacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacaagctgactctagcatgctaatacgactcactatagggcctttcaactgatgaggccgaaaggccgaaaacccggtatcccgggtcc

SEQ ID NO: 22 - protein, 219 aa, Foot-and-mouth disease virus
TTSAGEGAEPVTVDASQHGGNSRGVHRQHTDVSFLLDRFTLVGKTQNNKMTLDLLQTKEKALVGAILRAATYYFSDLEVACLGENKWVGWTPNGAPELEEVGDNPVVFSNRGATRFALPFTAPHRCLATTYNGDCKYKPAGTAPRDNIRGDLAVLAQRIAGETHIPTTFNYGRIYTEAEVDVYVRMKRAELYCPRPLLTHYDHNGKDRYKTAITKPAKQ

SEQ ID NO: 23 - protein, 659 aa, Foot-and-mouth disease virus
DKKTEETTLLEDRILTTSHGTTTSTTQSSVGITYGYADSDRFLPGPNTNGLETRVEQAERFFKHKLFDWTLEQQFGTTHVLELPTDHKGIYGQLVDSHSYIRNGWDVEVSATATQFNGGCLLVAMVPELCKLSEREKYQLTLFPHQFLNPRTNTTAHIQVPYLGVDRHDQGTRHKAWTLVVMVVAPYTNDQTIGSSKAEVYVNIAPTNVYVAGEKPAKQGILPVAVSDGYGGFQNTDPKTSDPVYGHVYNPARTGLPGRFTNLLDVAEACPTLLDFNGVPYVTTQANSGSKVLTCFDLAFGHKNLKNTFMSGLAQYYTQYSGTLNLHFMYTGPTNNKAKYMVAYIPPGTHPLPETPEMASHCYHAEWDTGLNSTFTFTVPYVSAADFAYTYSDEPEQASVQGWVGVYQVTDTHEKDGAVVVSVSAGPDFEFRMPISPSRQTTSAGEGAEPVTTDASQYGGDRRTTRRHHTDVSFLLDRFTLVGKTQDNRLTLDLLQTKEKALVGAILRAATYYFSDLEVACVGDNKWVGWTPNGAPELAEVGDNPVVFSKGGTTRFALPYTAPHRCLATAYNGDCKYKPTGTAPRENIRGDLATLAARIASETHIPTTFNYGRIYTDTVVDVYVRMKRAELYCPRPVLTHYDHGGKDRYKTAITKPVKQ

SEQ ID NO: 24 - protein, 221 aa, Foot-and-mouth disease virus
GIFPVAVSDGYGGFQNTDPKTSDPIYGHVYNPARTLYPGRFTNLLDVAEACPTLLDFNGVPYVQTQNNSGSKVLTCFDLAFGHKNMKNTYMSGLAQYFAQYSGTLNLHFMYTGPTNNKAKYMVAYIPPGTNPLPETPEMASHCYHAEWDTGLNSTFTFTVPYISAADYAYTYADEPEQASVQGWVGVYQITDTHEKDGAVVVSVSAGPDFEFRMPISPSRQ

SEQ ID NO: 25 - protein, 214 aa, Foot-and-mouth disease virus
TTSAGEGADVVTTDPSTHGGQVVEKRRMHTDVAFVLDRFTHVHTNKTTFNVDLMDTKDKTLVGALLRASTYYFCDLEIACVGDHQRVYWQPNGAPRTRELGDNPMVFSNKGVTRFAVPYTAPHRLLSTVYNGECKYETPVTAIRGDRAVLAAKYSNIKHTLPSTFNFGHVTADNSVDVYYRMKRAELYCPRPLFPAYDYASRDRFHVPIGVEKQ

(19) United States
(12) Patent Application Publication
(10) Pub. No.: US 2011/0014232 A1
(43) Pub. Date: Jan. 20, 2011

(54) CHIMERIC FOOT AND MOUTH DISEASE VIRUSES

(75) Inventors: Francois Frederick MAREE, Magalieskruin (ZA); Belinda Blignaut, Schoemansville (ZA); Aida Elizabeth Rieder, Westbrook, CT (US); Nicolaas Visser, Boxmeer (NL)

Correspondence Address: CLARK & ELBING LLP, 101 FEDERAL STREET, BOSTON, MA, 02110, US

(73) Assignees: Agricultural Research Council, Hatfield (ZA); United States of America as Represented by the Secretary of Agriculture, Washington, DC (US); Intervet International B.V., Boxmeer (NL)

(21) Appl. No.: 12/504,619

(22) Filed: Jul. 16, 2009

(51) Int. Cl. A61K 39/135; C07H 21/04; C12N 15/63; C12N 5/10; C12N 7/01

(52) U.S. Cl. 424/216.1; 536/23.72; 435/320.1; 435/352; 435/235.1; 536/25.3

(57) ABSTRACT

Foot and mouth disease (FMD) viruses which are able to grow on BHK-21 cells in suspension are described herein. The new viruses are recombinant chimeric viruses formed by replacing the outer capsid coding region of a first FMDV strain, which has previously been shown to be an effective vaccine strain, with the outer capsid coding region of a second FMDV strain. The outer capsid coding region of the second FMDV strain is also modified to introduce a heparan sulphate proteoglycan (HSPG) binding site. The chimeric viruses are then used as seed viruses in the production of inactivated vaccine antigens which have been tailored for specific outbreak situations or locality. The invention also relates to the product of expression of the chimeric FMD viruses and to uses therefor, such as to form antigenic, immunological or vaccine compositions for prevention of FMD.

### BACKGROUND OF THE INVENTION

The invention relates to chimeric foot and mouth disease viruses and to a method of constructing the chimeric virus. The method further relates to a method of eliciting an immune response to FMDV using the chimeric virus.

Foot-and-mouth disease (FMD) ranks as one of the most economically important infectious diseases of cloven-hoofed animals, affecting cattle, pigs, sheep, goats and other artiodactyl species. FMD is not only a disease affecting national and international trade, but impacts on the whole livestock industry with damaging consequences for the local farmer with invariable loss of income. Although eradicated from most parts of the world, FMD continues to occur in many developing countries where it severely constrains the livelihoods of poor livestock keepers. It seems unlikely that FMD will be eradicated from sub-Saharan Africa in the near future, because of the presence of large numbers of free-living maintenance hosts, particularly African buffalo. Livestock farming forms the backbone of rural economies for most of the SADC member countries. More than 75% of livestock is raised under the communal smallholder systems where it represents a multi-functional resource for the poor, providing meat, milk and fiber for household consumption or sale, traction for ploughing and transport, manure as fertiliser or fuel. Although mortality is usually low (less than 5%), FMD severely affects all of these functions as painful blisters in the mouth, feet and udder reduce livestock productivity, and the presence of the disease limits access to markets.

In southern Africa, the disease is essentially controlled through the separation of domestic and wildlife animals using fences, strategic vaccination of susceptible farm animals, restriction of animal movement and frequent inspections of animals and vaccination in controlled areas.

Movement restrictions and quarantine on animals and animal products during and after an outbreak severely impede trade, which is an important source of revenue for all income groups. Despite the fact that farmers are compensated if a stamping out policy is adopted, in many cases people are discouraged to continue producing livestock. Therefore, regular immunisation and improved vaccines, in terms of antigen yield, stability and protection against emerging FMD viruses (FMDV) are essential for disease control and maintaining the FMD-free status of South Africa.

FMDV is a naked icosahedral virus of about 25 nm in diameter, containing a single-stranded RNA molecule consisting of about 8500 nucleotides, with a positive polarity. FMDV exists as seven serologically distinct serotypes A, C, O, SAT (Southern African Territories) 1, 2, 3 and Asia 1. Although generally referred to as a single disease and clinically indistinguishable, the seven viral serotypes, distributed globally, have different geographical distributions and epidemiological profiles. The practical implication is that an animal infected with one serotype is not cross-protected and thus fully susceptible to infection by other FMDV serotypes. Six of the seven types of FMD virus, viz. SAT1, SAT2, SAT3, A, O and C, occur in sub-Saharan Africa. The fact that the SAT types are unique to Africa and have appreciably greater intratypic genomic and antigenic variation than the traditional “European” types, complicates FMD control in the subcontinent. SAT2 has the highest incidence in domestic animals in Africa causing more frequent outbreaks, while SAT1 viruses are recovered more frequently from carrier buffalo.

Vaccines are the most effective means of controlling and perhaps eventually eliminating infectious diseases, but existing FMD vaccines are not ideal. The effective administration and optimal induction of protective immunity against clinical disease are hampered by several factors. Vaccination against a specific serotype does not protect against the others. Even within a serotype distinct genetic and antigenic variants exist in different geographical regions with serious implications for the control of the disease by vaccination since it may render available vaccines inadequate. As an inactivated vaccine, it induces a short-lived immunity and animals have to be vaccinated twice annually. Vaccination does not prevent infection, it only delays the onset/progress of the disease and animals could become persistently infected, and in turn may be able to infect non-vaccinated animals. As it is problematic to distinguish between vaccinated and convalescent animals, the export market is lost for farmers in the FMD-controlled zone.

Commercial FMD vaccines are still classically produced by infection of cell culture by the virus followed by inactivation of the virus, usually by chemical treatment, e.g. with binary-ethylenimine (BEI). In order for FMD-virus vaccine production to be economically feasible, the FMD virus must be grown on cells in suspension, rather than cells attached to a monolayer. Therefore, classical FMD vaccines are limited to virus strains that are adapted to growth in cell cultures, most preferably suspension cell cultures.

Adaptation of new vaccine strains of FMDV up till now requires repeated passaging in cell cultures and depends on the acquisition of the capacity to bind cell-surface heparan sulphate, an alternative receptor for FMDV cell-entry. The acquisition of this capacity is totally dependent on random mutations and can therefore in no way be influenced. During the adaptation process, a virus isolate is first grown on, for example, primary pig or bovine epithelium cells, followed by adaptation on, for example, immortal pig kidney (IB-RS-2) and/or baby hamster kidney (BHK-21; ATCC-CCL-10) cells.

Cells grown in suspenson, e.g. suspension BHK-21 cells, are often insensitive to infection with wild-type FMDV for vaccine production, and thus the viruses have to be adapted to such cells before large scale production can commence.

This adaptation process for FMDV has two severe drawbacks:
The first drawback is that due to the random character of mutations, it is an unpredictable and thus time consuming process (it may easily take several months).
Another severe drawback is that during the process of repeated propagation, many other random mutations occur, during which the virus may undergo undesirable amino acid changes that may alter the antigenic determinants of the isolate.

The outcome may thus be an adapted vaccine strain that does not elicit a protective immune response against the parental virus or a vaccine strain that results in low or unstable antigen yield in large scale production.
As a consequence, it may be found that once the strain has aquired the capacity to bind heparin sulphate, it has lost its antigenic characteristics and thus is unsuitable for commercial vaccine production, and the process would need to begin from scratch with a new strain.

There is therefore a need to provide new FMD viruses which are more easily adapted to grow on BHK-21 cells, more specifically on BHK-21 cells in suspension, and are therefore ready to use in large scale production, allowing for fast and effective production of new vaccine strains.

### SUMMARY OF THE INVENTION

According to a first embodiment of the invention, there is provided a chimeric foot and mouth disease virus (FMDV) nucleic acid molecule encoding a first FMDV strain, wherein nucleotides encoding an outer capsid region have been replaced with nucleotides encoding an outer capsid region of a second FMDV strain which includes or has been modified so as to introduce a heparan sulphate proteoglycan binding site.

The first and second FMDV strains may be the same or different serotypes, independently selected from SAT1, SAT2, SAT3, A, C, O and Asia 1 serotypes.

The first FMDV strain is typically a strain which is able to grow in vitro on a commercial scale, and the second strain is typically a wild-type strain in current circulation.

The heparan sulphate proteoglycan binding site may be introduced by modifying one or more nucleotides of the outer capsid region of the second FMDV strain to encode:
a. lysine or arginine at residue 110 of SAT1 VP1 (SEQ ID NO: 22);
b. lysine or arginine at residue 112 of SAT1 VP1 (SEQ ID NO: 22);
c. lysine or arginine at residue 135 of SAT1 VP3 (SEQ ID NO: 24);
d. lysine or arginine at residue 175 of SAT1 VP3 (SEQ ID NO: 24);
e. lysine or arginine at residue 74 of SAT1 VP2 (SEQ ID NO: 23);
f. lysine or arginine at residue 83 of SAT2 VP1 (SEQ ID NO: 25);
g. lysine or arginine at residue 85 of SAT2 VP1 (SEQ ID NO: 25);
h. lysine or arginine at residue 161 of SAT2 VP1 (SEQ ID NO: 25); or
i. lysine or arginine at an equivalent position of one or more of (a)-(h) of another strain.

The nucleotides encoding amino acid residues at positions 110 and 112 of VP1 (SEQ ID NO: 22) or at positions 135 and 175 of VP3 (SEQ ID NO: 24) may be additionally modified to encode a lysine or arginine residue if the second FMDV is a SAT1 serotype.

The nucleotides encoding amino acid residues at positions 83 and 85 of VP1 or at position 161 of VP1 (SEQ ID NO: 25) may be additionally modified to encode a lysine or arginine residue if the second FMDV is a SAT2 serotype.

The first FMDV strain may have at least 70%, 80%, 90% or 95% sequence identity to SEQ ID NO: 1 or SEQ ID NO: 2, or an RNA equivalent thereof.

The capsid encoding region of the second FMDV strain may be a DNA or RNA sequence encoding the amino acid sequence of SEQ ID NOs: 3, 4 or 5, or a sequence which has at least 70%, 80%, 90% or 95% sequence identity thereto.

According to a further embodiment of the invention, there is provided a vector comprising a nucleic acid molecule described above.

According to a further embodiment of the invention, there is provided a host cell comprising a nucleic acid molecule described above. The host cell may be a BHK-21 cell.

According to a further embodiment of the invention, there is provided a virus expressed by or comprising the nucleic acid molecule described above. The virus may be inactivated.

According to a further embodiment of the invention, there is provided a composition comprising the virus or nucleic acid molecule described above. The composition may include a suitable adjuvant.

The composition may be used for eliciting an immune response against FMDV in a subject.

According to a further embodiment of the invention, there is provided a method of eliciting an immune response to FMDV in a subject, the method comprising administering the virus or the composition described above to the subject.

According to a further embodiment of the invention, there is provided a method of producing a chimeric FMDV nucleic acid molecule, the method comprising the steps of:
modifying a nucleotide sequence encoding an external capsid protein of a first FMDV strain to introduce a heparan sulphate proteoglycan (HSPG) binding site; and
inserting the modified capsid-coding nucleotide sequence into a nucleotide sequence of a second FMDV strain.

The modified capsid-coding nucleotide sequence of the first FMDV strain may replace nucleotides encoding the external capsid protein of the second FMDV strain.

Another nucleotide sequence encoding another capsid protein of the first FMDV strain may be additionally inserted into the second FMDV strain.

The first and second FMDV strains may be the same or different serotypes and may be independently selected from serotypes SAT1, SAT2, SAT3, A, C, O and Asia 1.

The heparan sulphate proteoglycan binding site may be introduced by modifying one or more nucleotides of the outer capsid region of the second FMDV strain to encode:
a. lysine or arginine at residue 110 of SAT1 VP1 (SEQ ID NO: 22);
b. lysine or arginine at residue 112 of SAT1 VP1 (SEQ ID NO: 22);
c. lysine or arginine at residue 135 of SAT1 VP3 (SEQ ID NO: 24);
d. lysine or arginine at residue 175 of SAT1 VP3 (SEQ ID NO: 24);
e. lysine or arginine at residue 74 of SAT1 VP2 (SEQ ID NO: 23);
f. lysine or arginine at residue 83 of SAT2 VP1 (SEQ ID NO: 25);
g. lysine or arginine at residue 85 of SAT2 VP1 (SEQ ID NO: 25);
h. lysine or arginine at residue 161 of SAT2 VP1 (SEQ ID NO: 25); or
i. lysine or arginine at an equivalent position of one or more of (a)-(h) of another serotype.

The nucleotides encoding amino acid residues at positions 110 and 112 of VP1 (SEQ ID NO: 22) or at positions 135 and 175 of VP3 (SEQ ID NO: 24) may be additionally modified to encode a lysine or arginine residue if the first FMDV strain is a SAT1 serotype.

The nucleotides encoding amino acid residues at positions 83 and 85 of VP1 or at position 161 of VP1 (SEQ ID NO: 25) may be additionally modified to encode a lysine or arginine residue if the first FMDV strain is a SAT2 serotype.

### BRIEF DESCRIPTION OF THE FIGURES

FIG. 1 Shows a schematic diagram of the pSAT1 and pSAT2 genome-length clones. The complete genome was cloned under control of a T7 promotor in a pGEM based vector and ended with a plasmid coded T7 terminator. The 5′ and 3′ ends of the genome are flanked by hammerhead (HH) and hepatitis delta virus (HDV) ribozymes to allow generation of the correct termini of recombinant viral RNA. The complete genome, including 5′ and 3′ untranslated regions and poly A tail are present. The complete vector sequences are shown in FIG. 7.

FIG. 2 Shows a schematic diagram of the cloning strategy for engineering chimera viruses. Chimeric SAT viruses were constructed by replacement of the external capsid coding region, 1B/1C/10, of pSAT1 or pSAT2 by the corresponding region of the selected field isolates. The genetically engineered SAT chimeras contain the capsid of the field viruses.

FIG. 3 Shows the plaque morphologies of SAT1 and SAT2 vaccine strains (high passage) and the parental isolates (low passage). Field strains were also repeatedly passage on BHK-21 cells until the adaptation was achieved.

FIG. 4 Shows the 3D structure of SAT1/SAR/9/81 (A) and SAT2/ZIM/7/83 (B) capsid crystallographic protomers modelled using the O1BFS co-ordinates (1FOD) as template. (A) The position of amino acid substitutions found in high BHK-21 passage SAT1 and SAT2 viruses compared to the field isolates are indicated as black spheres. VP1 is represented in dark grey, VP2 in light grey and VP3 in medium grey.

FIG. 5 Shows the plaque morphologies of chimeric viruses containing the wild-type outer capsid proteins of SAT1/NAM/307/98 (A; vNAM/SAT) and SAT2/SAU/6//00 (C; vSAU/SAT) cloned into the genetic background of pSAT2. The change in plaque phenotype on BHK-21 cells and the susceptibility of CHO—K1 cells for infection by the mutant vNAMΔKRR (B) is shown. The mutant vSAUΔHRR (D) and pSAUΔKHK (E) displayed the same plaque morphology than the wild-type chimera and did not grow in CHO—K1 cells.

FIG. 6 Antibody response elicited in pigs by full (6.0 μg), quarter (1.5 μg) and one-sixteenth (0.375 μg) doses of the vKNP/SAT2 chimera (A) and SAT1/KNP/196/91 parental (B) vaccines, respectively.

FIG. 7 Shows the complete vector sequences (SEQ ID NOs: 20 and 21) containing the (i) SAT2 and (ii) SAT1 genome-length cDNA (SEQ ID NOs: 1 and 2; non-italics).

FIG. 8 Shows the amino acid sequences of the capsid proteins of FMDV strains used in the identification of heparin sulfate proteoglycan binding sites: (i) SAR/09/81 Impala Epith (SEQ ID NO: 8); KNP/196/91 PK1 (SEQ ID NO: 9); NAM/307/98/1 PK1RS4 (SEQ ID NO: 10); ZAM/2/93 PK1RS3 (SEQ ID NO: 11); KNP/19/89 PK1RS2 (SEQ ID NO: 12); ZIM/07/83/2 (SEQ ID NO: 13); ZIM/05/83 BTY4RS1 (SEQ ID NO: 14); ZIM/14/90/2 BTY1RS3 (SEQ ID NO: 15); ZAM/7/96 BTY1RS2 (SEQ ID NO: 16). The sequences of the primary isolates are shown and the substitution observed during adaptation on BHK-21 cells summarized in Table 2. SAT1 VP1 is shown in (i) in italics (SEQ ID NO: 22); SAT1 VP2 is shown in (ii) in italics (SEQ ID NO: 23); SAT1 VP3 is shown in (iii) in italics (SEQ ID NO: 24); and SAT2 VP1 is shown in (v) in italics (SEQ ID NO: 25). The residues for modification are shown in bold.

FIG. 9 Shows the amino acid sequences of the capsid protein of three chimeric viruses. The sequences for the outer capsid proteins of (i) SAT1/KNP/196/91 (SEQ ID NO: 3), (ii) SAT1/NAM/307/98 (SEQ ID NO: 4) and (iii) SAT2/SAU/6/00 (SEQ ID NO: 5) (shown in normal font) were inserted into the corresponding region of pSAT2 (shown in italics) (SEQ ID NOs: 6 and 7). Viable chimeric viruses, i.e. vKNP/SAT, vNAM/SAT and vSAU/SAT (SEQ ID NOs: 17-19) were generated. These constructs were used for the insertion of HSPG-binding residues (in bold).

### DETAILED DESCRIPTION OF THE INVENTION

New foot and mouth disease (FMV) viruses which are able to grow on BHK-21 cells in suspension (and which therefore do not need to undergo the time-consuming and possibly immunogenicity-destroying adaptation process) are described herein. As they are immediately able to grow on BHK-21 cells in suspension, they are ready for use in the large scale production of vaccines.

The new viruses are recombinant chimeric viruses formed by replacing the outer capsid coding region of a first FMDV strain which has previously been shown to be an effective vaccine strain with the outer capsid coding region of a second FMDV strain. The outer capsid coding region of the second FMDV strain is also modified to introduce a heparan sulphate proteoglycan (HSPG) binding site. The chimeric viruses are then used as seed viruses in the production of inactivated vaccine antigens which have been tailored for specific outbreak situations or locality.

These chimeric viruses, which contain the antigenic determinants of a field virus, do not need to undergo the time consuming and expensive adaptation process in order to be grown in vitro to large scale. Also, as the virus does not need to undergo numerous passages, uncertainty about final antigen yields and characteristics can be avoided.

The invention also relates to the product of expression of the chimeric FMD viruses and to uses therefor, such as to form antigenic, immunological or vaccine compositions for prevention of FMD.

The chimeric viruses, vectors containing them or expression products, such as antigens, can be administered to a subject to prevent FMD. The subject can be any animal which can become infected with FMDV, in particular, bovine, ovine, porcine or caprine species.

The chimeric viruses, vectors or expression products thereof, or immunological, antigenic or vaccine compositions containing them, are typically administered via a parenteral route (intradermal, intramuscular or subcutaneous). Such an administration enables a systemic immune response, or humoral or cell-mediated responses.

The compositions contemplated by the invention can also contain an adjuvant. Suitable adjuvants are well-known in the art.

The use of infectious cDNA technology in synthesising vaccines for specific geographic localities or an outbreak situation against emerging or contemporary virus strains has previously been described (Rieder et al., 1993; Zibert et al., 1990; Almeida et al., 1998; Beard and Mason, 2000; van Rensburg et al., 2004; Fowler et al., 2008). Viable genome-length cDNA clones have been applied successfully in recent years in studying the biological properties of FMDV. The cDNA clones can be manipulated by genetic engineering techniques, exchanging genome segments or introducing single nucleotide changes and still rendering viable chimeric viruses following transfection and successive passages in vitro.

Infectious genome-length cDNA clones of SAT1 and SAT2 strains were constructed with the desirable biological properties of good vaccine strains (van Rensburg et al., 2004). The antigenic characteristics of such a clone can then be manipulated by merely exchanging the determinants of antigenicity, i.e. the structural proteins or parts of it (Rieder et al., 1993; Sa-Carvalho et al., 1997; Almeida et al., 1998; Beard and Mason, 2000; van Rensburg et al., 2004). The fact that the viral RNA can be made infectious in the absence of other components of the virion allows the recovery of genetically engineered new viruses from in vitro-generated RNA molecules (Zibert et al., 1990).

The chimera technology can be applied in the development of custom-made vaccines specific for a geographical region. The applicants used a chimera virus containing the outer capsid proteins of a SAT1 virus, cloned into a SAT2 genetic background, to vaccinate animals in a full potency trial and observed similar protection compared to the parental SAT1 vaccine. In the construction of a chimera, the cell-entry determinants, like the antigenicity, of the field isolate are transferred to the derived chimeric virus.

A major factor that is likely to contribute to the poor growth of field viruses in cell culture is the lack of appropriate host-specific integrin receptors for virus cell-attachment. Cultivation of FMDV in cultured BHK-21 cells leads to the adaption of FMDV for growth in cell culture and can select for variants with a high affinity for HSPGs (glycosaminoglycans or GAG's). This phenotype, and consequently an ability to use HSPGs as alternative receptors to initiate infection, is associated with the accumulation of positively charged residues in surface-exposed loops of the outer capsid proteins. HSPG receptors are found on most cell surfaces. This is a major advantage for vaccine manufactures, as HSPG-binding results in an expanded host range for cultured cells and permits the use of established cell lines, like BHK-21 cells, in suspension in fermentors. Heparan sulphate binding sites are described in more detail in Fry et al. (Embo J., Vol 18, pp 543-554, 1999). The downside of this adaptation process has been discussed above, specifically in relation to the disadvantages of random mutagenesis affecting the antigenic characteristics. These disadvantages could, however, not be avoided until now.

The applicants have now identified unique HSPG-binding sites (amino acid residues) located on the outer capsid proteins of SAT1 and SAT2 FMDV. The sites are exposed on the surface of the virion and are structurally accessible for binding to the alternative HSPG receptors.

These binding sites were identified on FMDV isolates (vaccine strains) that have adapted to growth on BHK-21 cells (ATCC-CCL-10) in suspension, a cell used in the production of FMDV vaccines, by comparing the amino acid sequences of current SAT1 and SAT2 vaccine strains with the corresponding primary isolates, available at Transboundary Animal Diseases Program (TADP) of the ARC-OVI (Onderstepoort Veterinary Institute, South Africa). The vaccine strains also have the ability to infect and replicate in Chinese hamster ovary cells strain K1 (CHO—K1 ATCC CCL-61) cells, a feature characteristic of viruses that use HSPG receptors for cell entry. The residue substitutions were located on surface-exposed loops and included a change to a positive charge residue. These binding sites were shown to simultaneously affect plaque phenotype, virus host range in cell culture and the ability to infect cells in culture via HSPG.

The invention is illustrated in more detail in the Example section, below, for two of the most distantly related FMDV-viruses; SAT 1 and SAT 2. However, it is emphasized that the same approach is perfectly and without undue burden applicable to SAT 3, A, O, C and Asia I serotypes.

The eight novel amino acid positions/sites on the outer capsid proteins of SAT1 and SAT2 viruses identified by the applicants are typically associated with changes observed in the plaque morphology on BHK21 cells, infection and replication of CHO—K1 cells and the ability to utilise HSPG for cell entry. CHO—K1 cells do not express the integrins that facilitate cell entry of FMDV and infection is via HSPG receptors. This characteristic is also associated with the ability of FMDV to infect BHK-21 cells in suspension. Five of the eight amino acid positions were specifically identified on SAT1 isolates and the remaining three on the SAT2 serotype.

The sites in SAT1 viruses included a (1) lysine or arginine at residue 110 of VP1, (2) lysine or arginine at residue 112 of SAT1 VP1, (3) lysine or arginine at residue 135 of VP3, (4) lysine or arginine at residue 175 of VP3, (5) lysine or arginine at residue 74 of VP2. The position of the sites prone to change during adaptation of SAT2 viruses was a (6) lysine or arginine at residue 83 of VP1, (7) lysine or arginine at residue 85 of VP1, (8) lysine or arginine at residue 161 of VP1. Residues 110-112 of VP1 seem to be a “hotspot” for change in SAT1 viruses during cell culture adaptation, since three viruses with substitutions at this position were identified, i.e. SAR/9/81, KNP/196/91 and ZAM/2/93. Similarly, the residues 83 and 85 were prone to change during adaptation of two SAT2 viruses, i.e. KNP/19/89 and ZAM/7/96. These novel HSPG-binding sites have been shown to improve the cell-entry and replication ability of SAT1 and SAT2 isolates in BHK-21 monolayers or suspension cultures, which are characteristics sought after in a good vaccine strain.

The novel amino acid substitutions identified by the applicants during adaptation of SAT viruses (like vaccine strains) on BHK-21 cells can be engineered into new vaccine strains using recombinant DNA technology. Introducing the identified HSPG-binding sites when constructing a chimeric virus from a field isolate can similarly improve the cell-entry mechanism and result in a virus that is immediately adapted for large scale production in suspension cells. This allows for fast and effective adaptation of recombinantly generated new vaccine strains from an isolate in an outbreak situation or specific geographic location. The engineered HSPG-binding virus can be amplified within a few passages directly on BHK-21 to create a master seed stock, without prior isolation on primary cell lines and further adaptation.

The HSPG binding regions can be used in combination with recombinant chimeric technology. In particular, the outer capsid-coding region from a genome-length cDNA clone can be exchanged with the corresponding region of a field isolate. The virus recovered from such a chimeric DNA construct will have features from both the recombinant genetic backbone and the field isolate. For SAT1 serotypes, a lysine or arginine can be simultaneously introduced at positions 110 and 112 of VP1 or a lysine or arginine can be simultaneously introduced at positions 135 and 175 of VP3 of the wild-type sequence via site-directed mutagenesis. The new SAT1 recombinant chimeric virus can be multiplied to generate vaccine seed virus for large scale production of the chimeric SAT1 inactivated vaccines. Similarly, the HSPG-binding sites, a lysine or arginine can be simultaneously inserted at positions 83 and 85 of VP1 or at position 161 of VP1 in a wild-type SAT2sequence. The SAT2 chimeric virus can be used to generate vaccine seed virus. Custom-made vaccines from isolates from a specific outbreak situation or geographic region can be produced according to this method.

The present invention is further described by the following examples. Such examples, however, are not to be construed as limiting in any way either the spirit or scope of the invention.

### Examples

Infectious Genome-Length cDNA Technology

A genome-length cDNA copy (pSAT2) of the SAT2 vaccine strain, ZIM/7/83, was constructed following an exchange-cassette strategy using an Al 2 genome-length clone as template (Rieder et al., 1993; van Rensburg et al. 2004). The SAT2/ZIM/7/83 virus contained all the characteristics of an ideal vaccine candidate, including fast growth properties, high antigen yields and a broad antigenic coverage. This initial construct was used for the transfection of in vitro synthesized RNA transcripts, followed by the recovery of infectious viral particles. Through manipulation of this clone, in particular the inclusion of hammerhead and hepatitis delta virus ribozymes, a pSAT2r+ clone was generated that could be utilised for the production of viable viruses by direct transfection of baby hamster kidney (BHK-21) cells with DNA, eliminating the time consuming process of RNA synthesis in vitro.

Using similar cloning methodology previously described (van Rensburg et al. 2004), a genome-length cDNA copy of the SAT1 vaccine strain, SAR/9/81, was also constructed (designated pSAT1). A SAT1 strain (SAR/9/81) isolated from impala epithelium (SAR/9/81imp) and the tissue culture adapted version (SAR/9/8lvacc; PK1RS4-BHK5) were selected to facilitate the construction of the clones. The SAR/9/81 virus was selected for its favourable growth properties, easy adaptation on tissue culture cells, i.e. IB-RS2 and BHK-21 cell lines, and excellent vaccine strain properties.

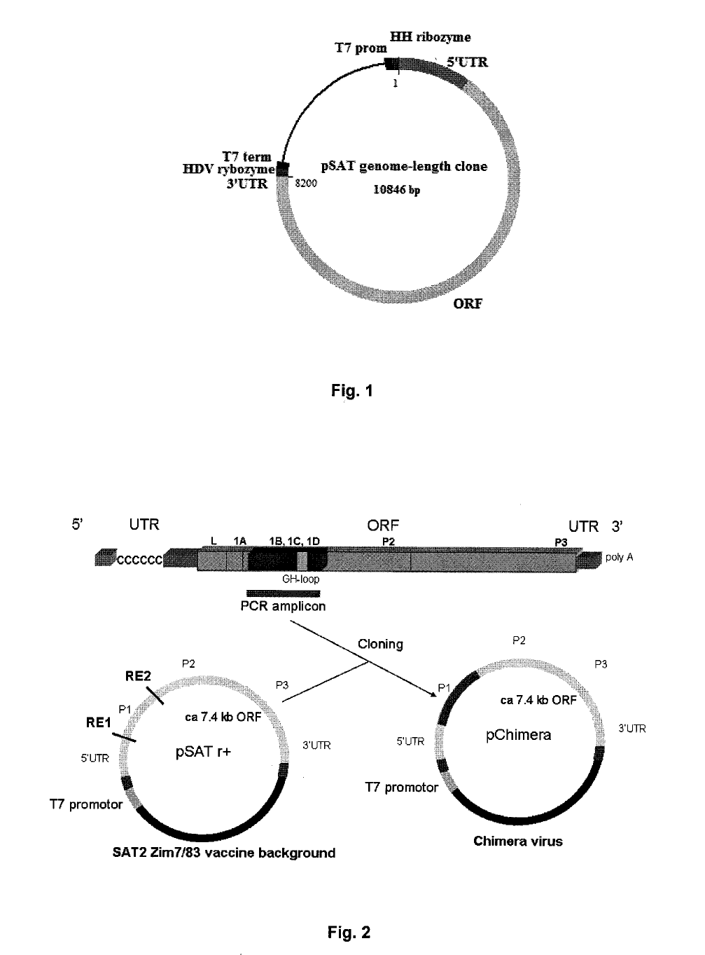

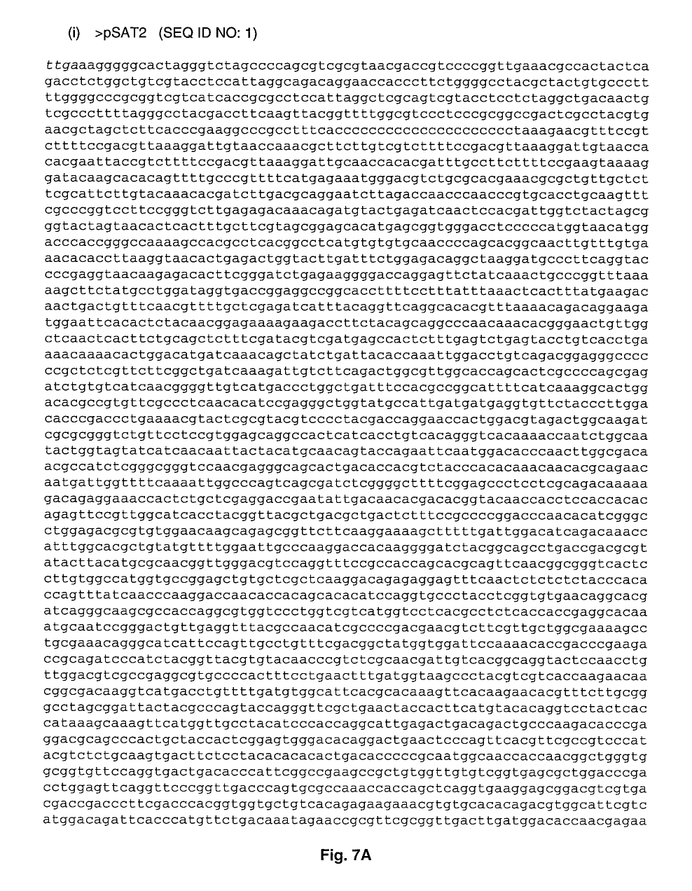

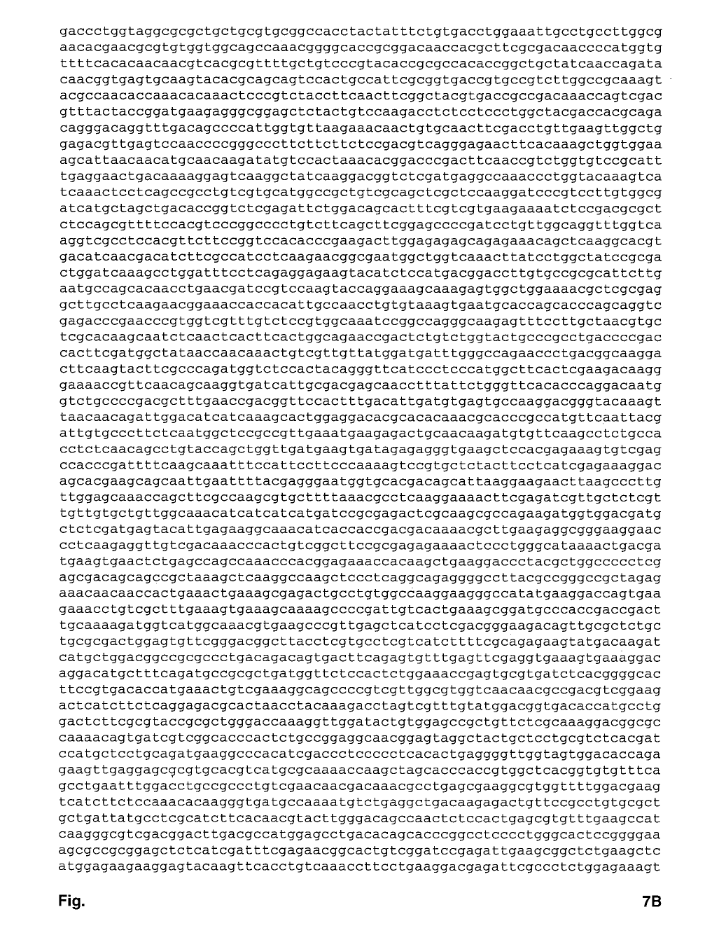

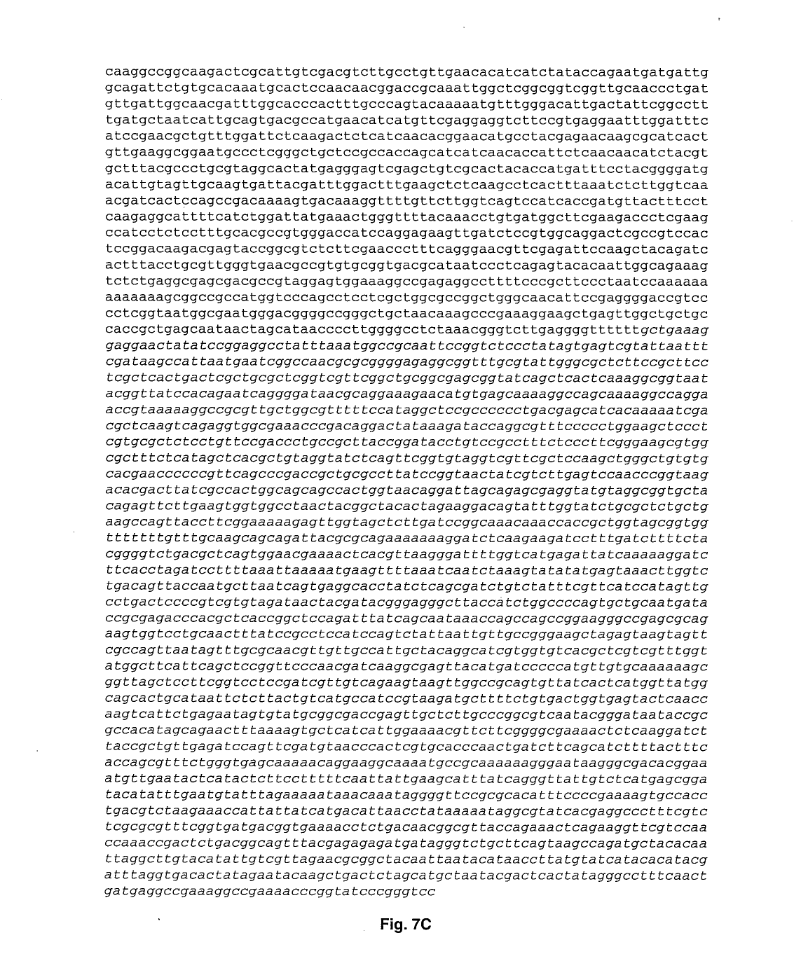

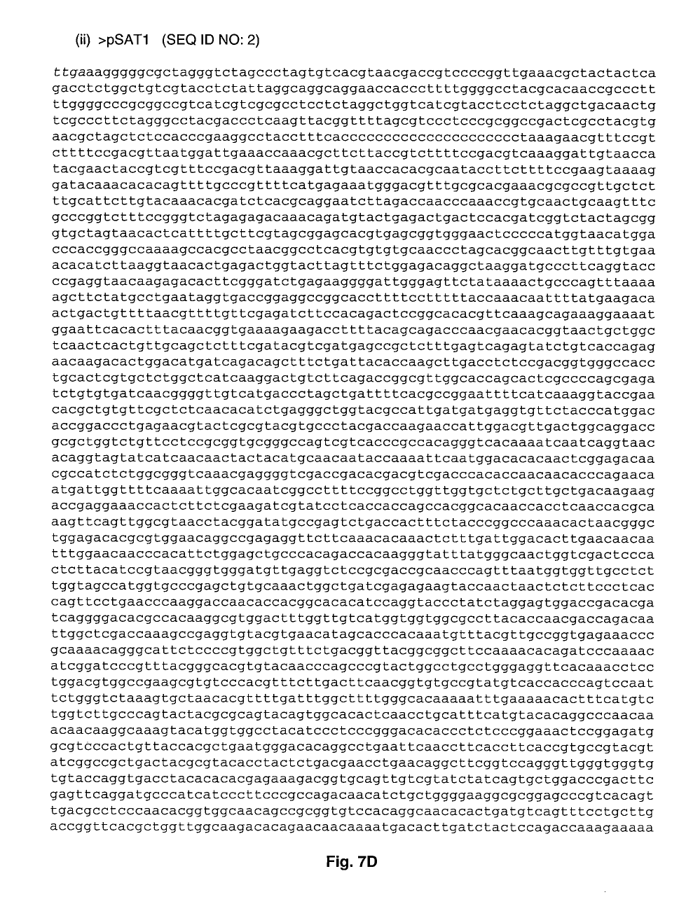

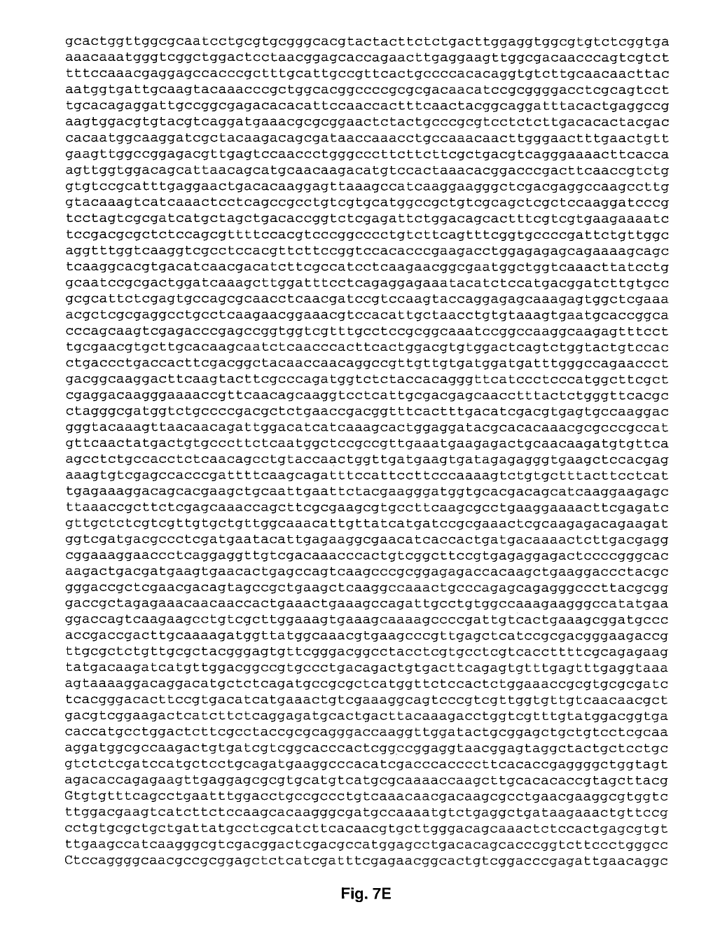

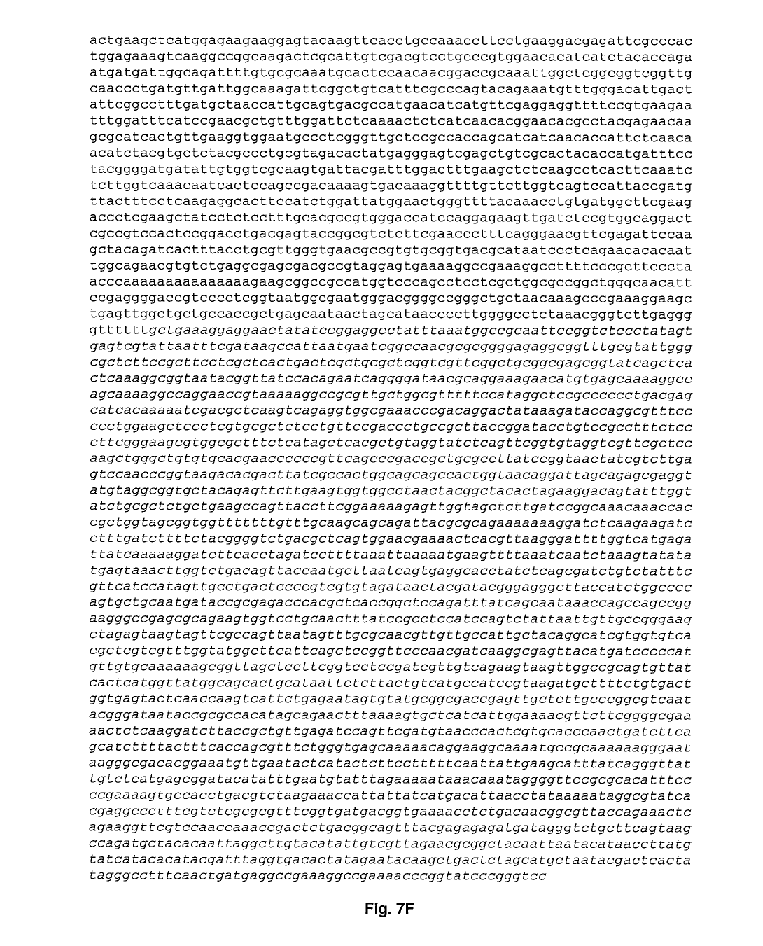

The importance of the pSAT1 clone stems from the fact that the FMDV serotypes are antigenically diverse and very little or no cross-protection exists between serotypes. Also, the outer capsid proteins of SAT1 viruses are seven amino acids longer than for SAT2. Both the pSAT1 and pSAT2 vectors can be used to prepare synthetic RNA, which in turn is used to transfect BHK-21 cells. The general vector map for pSAT1 and pSAT2 is depicted in FIG. 1 and the vector sequences are shown in FIG. 7.

Both the pSAT1 and pSAT2 clones were modified by introduction of novel restriction enzyme (RE) sites to allow the exchange of the outer capsid-coding region with the corresponding region of contemporary viruses. The RE sites for sspI (ATTAAT (SEQ ID NO: 17)) and xmaI (CCCGGG (SEQ ID NO: 18)) were introduced, while natural occurring xmaI sites in the pSAT1 were removed, by standard site-directed mutagenesis protocols (Sambrook and Russel., 2001).

The method of the present invention has been shown to work equally well for divergent serotypes of FMDV, and is easily applicable to other serotypes not specifically exemplified herein.

Application of Infectious cDNA Clones and the Construction of Chimeric Viruses

The outer capsid-coding regions of pSAT1 and pSAT2 were replaced with that of SAT1, SAT2 and SAT3 field and vaccine strains. Basic cloning methodology as described in Sambrook and Russel, 2001 was used. The genome-replacement strategy is illustrated in FIG. 2.

The applicants were able to construct a panel of viable chimeric viruses from the pSAT2 and pSAT1 genome-length cDNA clones by replacing the external capsid-coding region with the corresponding region from SAT1, 2 and 3 vaccines strains and/or field isolates. The resulting chimeras showed growth characteristics and immune profiles comparable to the parental viruses used for the cloning process, indicating that the derived chimeras were similar to the field strains. In many instances, the chimeras represented a subpopulation of the field strains as a result of the quasispecies nature of FMDV, and in at least one instance the biological properties of the field isolate were improved by the presence of the encoded replication determinants of the genome-length backbone. The cell-receptor binding preference of the field isolates was retained in the chimeric viruses.

The SAT field strains that were selected for the chimeras are summarised in Table 1 and included three SAT2 strains from the southern African topotype (ZIM/17/90, ZIM/14/91 and ZAM/07/96), two SAT1 viruses (NAM/307/98 and ZAM/02/93) and a SAT3 virus (ZAM/04/96). The external capsid-coding regions of 6 field strains were recovered via PCR amplification, introducing unique restriction enzyme sites to facilitate cloning (FIG. 2). The corresponding region from pSAT2r+ was removed and replaced by the field strains external capsid-coding amplicons.

The applicants have shown that the antigenic determinants of the field isolates are transferrable to the recombinant synthesized chimeric virus. Similarly, the receptor preference and inability to enter cultured cells via HSPG receptors of the field isolates was also transferred to the chimera viruses. The chimera technology for the production of vaccines specific for geographic locality or outbreak situation can be refined by introducing HSPG-binding sites during the construction of the chimera.

TABLE 1
Summary of the viruses which were used in the construction of chimeric
viruses, and their history. The amino acid differences between the 1B/C/D-2A
chimeric viruses and the parental isolates are also indicated.

| | Species | | | | |
|---|---|---|---|---|---|
| | isolated | Passage | Country of | | Chimeric virus^{b} and |
| FMDV strain | from | history | origin | Topotype^{a} | genetic backbone^{c} |
| SAT1/KNP/196/91 | Buffalo | PK1RS1 | Kruger Nat | 1 | pKNP/SAT2 |
| | | | Park | | |
| SAT1/NAM/307/98 | Buffalo | PK1RS4 | Namibia | 2 | pNAM/SAT2 |
| SAT1/ZAM/2/93 | Buffalo | PK1RS3 | Zambia | 3 | pZAM/SAT2 |
| SAT2/ZIM/14/90 | Buffalo | BTY1RS3 | southern | II | vZIM14/SAT2 |
| | | | Zimbabwe | | |
| SAT2/ZIM/17/91 | Buffalo | BTY2RS4 | southern | II | vZIM17/SAT2 |
| | | | Zimbabwe | | |
| SAT2/ZAM/7/96 | Buffalo | BTY1RS2 | Zambia | III | vZAM7/SAT2 |
| SAT2/SAU/6/00 | Cattle | BTY1RS2 | Saudi Arabia | VIII | vSAU/SAT2 |
| SAT3/ZAM/4/96 | Buffalo | BTY1RS1 | Zambia | 4 | vZAM4/SAT2 |

^{a}Topotypes refers to genotypes distributed to specific geographic regions and the topotypes for the SAT serotypes are described by Bastos et al., 2001 and Bastos et al., 2003a, b.
^{b}Viruses recovered by transfection of BHK-21 cells are designated “v” followed by the parental isolate number and the SAT2 plasmid used for cloning purposes.
^{c}The genome-length clone used for the construction of the chimera is indicated after “/” in the designated name.

Mapping of Heparan Sulfate Binding Regions of SAT1 and SAT2 Virions

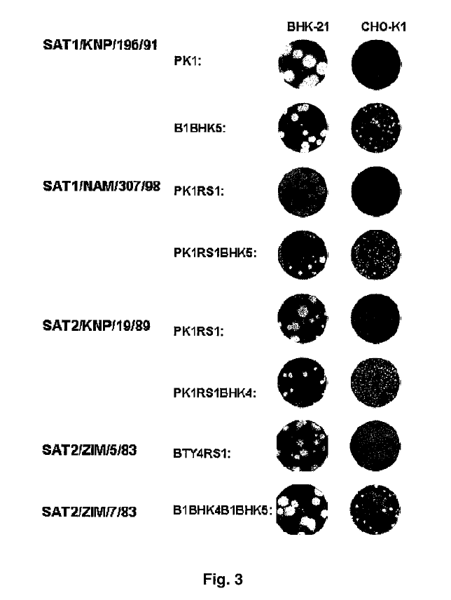

At least four SAT1 and four SAT2 viruses, grown to high passage in BHK-21 cells, were used in the study. The viruses include isolates that are currently in use in the preparation of inactivated vaccines at ARC-OVI, and these were compared to the parental isolates (low passage) from which they were derived. The virus isolates included SAT1/SAR/9/81, SAT1/KNP/196/91, SAT1/ZAM/2/93 and SAT1/NAM/307/98 from the SAT1 serotype and SAT2/KNP/19/89, SAT2/ZIM/7/83 (parental is labeled SAT2/ZIM/5/83), SAT2/ZIM/14/90 and SAT2/ZAM/7/96 of SAT2 serotype. The plaque phenotypes and cell culture host range of the high and low passage isolates of the abovementioned viruses were compared (FIG. 3). Sequence data was collected from the outer capsid-coding (P1) region for the abovementioned isolates and their derivatives (high passage and low passage viruses). Sequence variation within the non-coding regions of the genome and the non-structural coding regions are unlikely to influence receptor preference following adaptation in cell culture. Therefore, only amino acid changes within the capsid-coding regions of the genome were investigated.

In FIG. 3, the plaque phenotypes of primary isolates and cell culture-adapted SAT1 and SAT2 viruses in BHK-21 and CHO—K1 cells are shown. In general, the plaques of the primary isolates (low passage) of SAT1 viruses on BHK-21 cells are large (7-10 mm diameter), more homogeneous in nature and plaque edges are opaque. CHO—K1 cells were not able to sustain infection by the low passage SAT1 viruses, a feature associated with the inability of the virus to utilize the alternative HSPG for cell entry. On the contrary, the high BHK-passage viruses were characterised by a mixture of large, medium (4-6 mm) and small (1-2 mm) plaques, often with clear edges, and were accompanied with the ability to infect and replicate in CHO—K1 cells, indicative of the presence of the adaptation phenotype in all four SAT1 isolates. Similarly, the plaque morphology for the low passage SAT2 viruses consisted of medium to large plaques with opaque edges and the inability to infect and replicate in CHO—K1 cells, while the high passage viruses produced plaques with clear edges and successfully infected CHO—K1 cells.

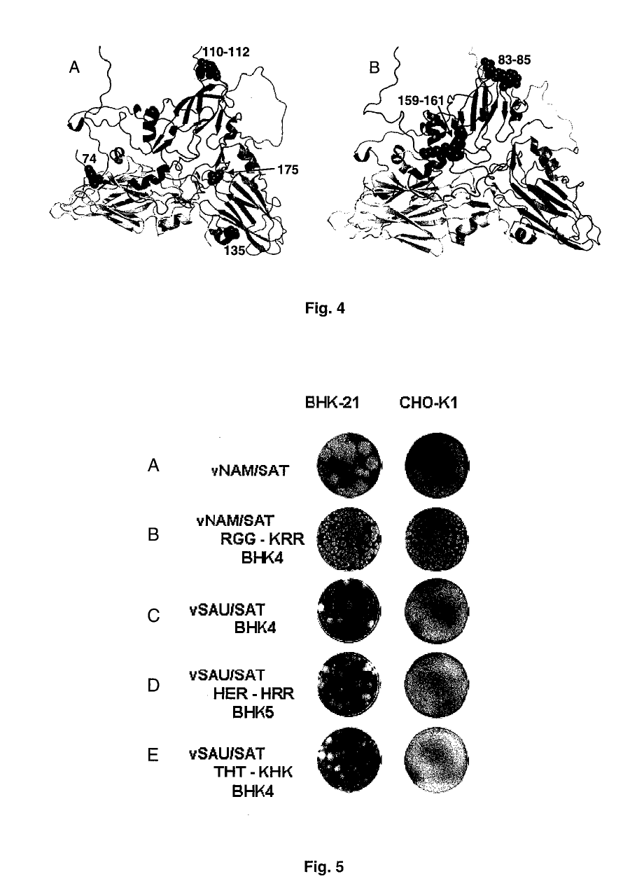

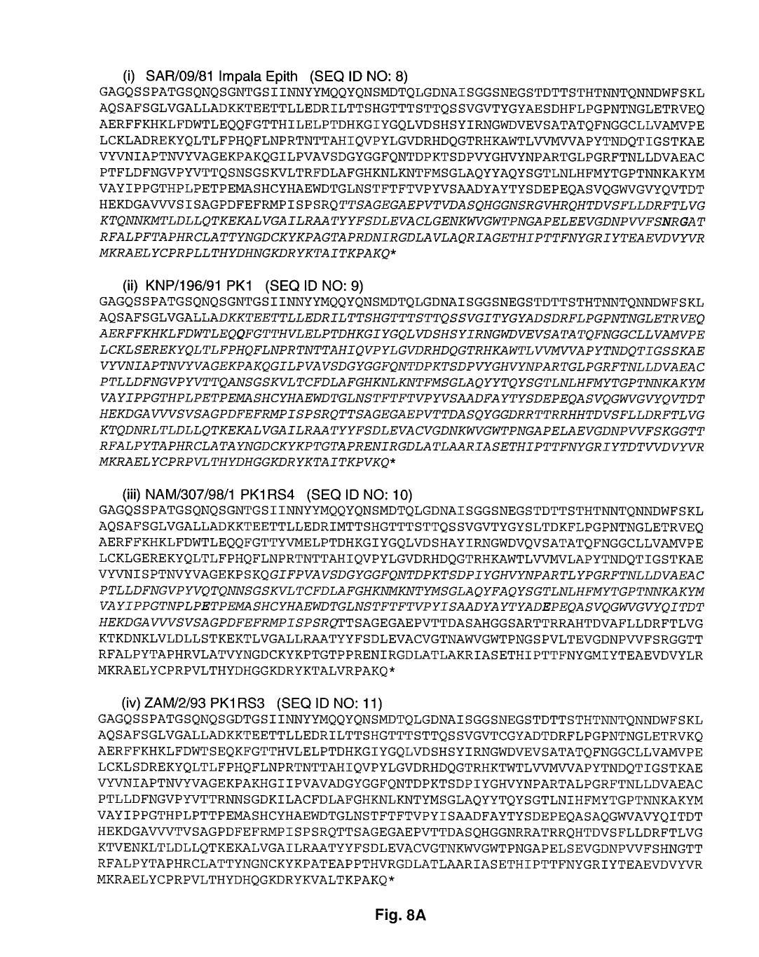

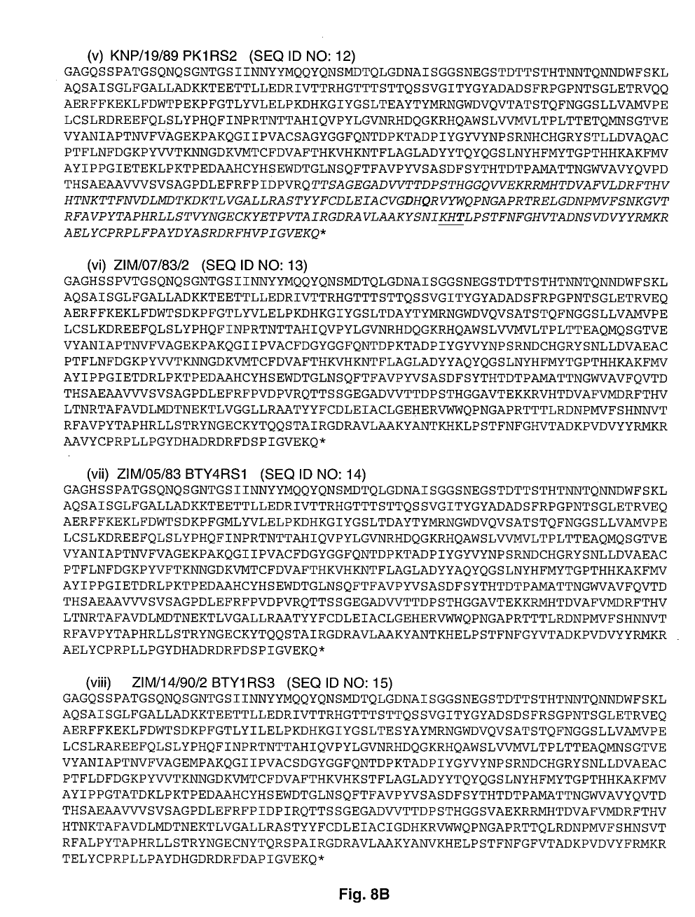

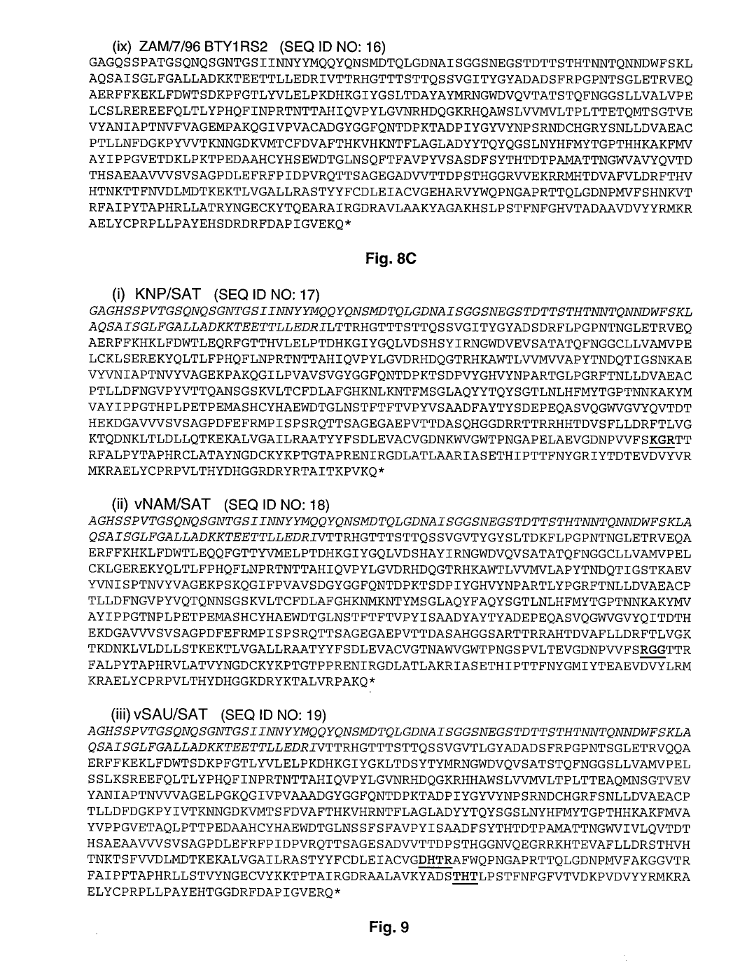

The nucleotide sequences of the outer capsid-coding regions were determined and the deduced amino acid sequences were compared (FIG. 8). Genotypically, the high passage viruses contained amino acid substitutions in the capsid proteins when compared to the low passage counterparts (Table 2). Sequence analysis, however, revealed multiple sites of amino acid differences, as can be expected from the quasispecies nature of FMDV. To determine the conformational location of the substitutions and identify those residues involved in cell culture adaptation and HSPG recognition, the amino acids were mapped to the structure of SAT1 and SAT2 virion (FIG. 4). The structures of the outer capsid proteins were modelled using MODELLER v7 and the X-ray crystallographic coordinates of O1BFS (PDB reference 1FOD).

TABLE 2
Comparison of the capsid forming amino acid sequences of SAT1 viruses and their cell culture
adapted derivatives

| | | aa variation & | | | | | | | | | | | | | | | |
|---|---|---|---|---|---|---|---|---|---|---|---|---|---|---|---|---|---|
| | | β-β struct | | | | | | | | | | | | | | | |
| | | SAT1: | | | | | | | | | | | | | | | |
| | | | | | | | | | | | | | | VP1 | | | |
| Virus | Passage | | VP2 | | | | | VP3 | | | | | | 1110- | | | |
| strain | history* | 2074 | 2134 | 2170 | 2196 | 3009 | 3135 | 3175 | 3192 | 3217 | 1018 | 1049 | 1069 | 1112 | 1179 | 1206 | 1210 |
| SAR/9/81 | Impala epi. | — | — | — | — | — | — | — | Asp | Ser | — | — | Ala | Asn- | — | — | — |
| | | | | | | | | | | | | | | Arg- | | | |
| | | | | | | | | | | | | | | Gly | | | |
| | PK1BHK5 | — | — | — | — | — | — | — | Tyr | Iso | — | — | Gly | Lys- | — | — | — |
| | | | | | | | | | | | | | | Arg- | | | |
| | | | | | | | | | | | | | | Arg | | | |
| KNP/196/91 | PK1 | Gln | Glu | Gln | Ser | Asp | — | — | — | — | Tyr | Arg | — | Lys- | Val | Lys | Lys |
| | | | | | | | | | | | | | | Gly- | | | |
| | | | | | | | | | | | | | | Gly | | | |
| | B1BHK5 | Arg | Asp | His | Gln | Val | — | — | — | — | His | Lys | — | Lys- | Glu | Arg | Arg |
| | | | | | | | | | | | | | | Gly- | | | |
| | | | | | | | | | | | | | | Arg | | | |
| NAM/307/98 | PK1RS1 | — | — | — | — | — | Glu | Glu | — | — | — | — | — | — | — | — | — |
| | PK1RS1BHK5 | — | — | — | — | — | Lys | Lys | — | — | — | — | — | — | — | — | — |
| ZAM/2/93 | PK1RS1 | — | — | — | — | — | — | — | — | — | — | — | — | His- | — | — | — |
| | | | | | | | | | | | | | | Asn- | — | — | — |
| | | | | | | | | | | | | | | Gly | | | |
| | PK1RS2BHK3 | — | — | — | — | — | — | — | — | — | — | — | — | Lys- | — | — | — |
| | | | | | | | | | | | | | | Asn- | — | — | — |
| | | | | | | | | | | | | | | Arg | | | |

*B, bovine; PK, primary pig kidney cells; RS, IB-RS2 cells; BHK, baby hamster kidney cells; Bovine thyroid cells; the number indicates the number of passages on the cell line in question

Binding of viruses to HSPG or other glycosaminoglycans (GAG) occurs mainly through electrostatic interactions between positively-charged Lys and Arg groups on the virus surface and the negatively-charged N and O sulphated groups of the GAG molecules (Gromm et al., 1995; Byrnes and Griffin, 1998 and 2000). The accumulated positively-charged residues and increased affinity to HS probably lead to direct interaction between the Arg or Lys and heparin. The selection of positively-charged residues was previously reported for type O viruses (Sa-Carvalho et al., 1997; Jackson et al., 1996; Zhao et al., 2003). Adaptation of O1 Campos to cell culture selected viruses with an H→R change at position 56 of VP3 (Jackson et al., 1996; Sa-Carvalho et al., 1997).

Therefore, in the investigation of residues or sites involved in HSPG binding, attention was placed on where the adaptation-to-suspension-cell-culture phenotype was accompanied by the acquisition of positive charged amino acid residues on surface exposed loops in the VP1, VP2 and VP3-capsid proteins (FIG. 4). The first line of evidence was seen with the recombinant vSAT1 virus, derived from the SAT1 genome-length clone. In FIG. 5, the recombinant virus displayed a small plaque phenotypic variant of the mixed plaque size population of the cell culture-adapted SAR/9/81 virus (high passage). The well-defined small plaque phenotype was consistent with adaptation of SAR/9/81 virus to cell culture. The homogeneity of the vSAT1 plaque phenotype provided the advantage of a direct correlation between genotype and phenotype. Genetically, the vSAT1 did not have a perfect match to the majority population of SAR/9/81 high passage virus it was derived from, an observation consistent with the quasispecies nature of the FMDV genome. The four amino acid differences in the pSAT1 clone outer capsid-coding region were detected in “pure” populations of the SAR/9/81 (Table 3).

TABLE 3
Comparison of the capsid forming amino acid sequences of SAT2 viruses and their cell culture
adapted derivatives

| | | SAT2: | | | | | | | | | | | | | | | | | | | | |
|---|---|---|---|---|---|---|---|---|---|---|---|---|---|---|---|---|---|---|---|---|---|---|
| | | | | VP2 | | | | | VP3 | | | | | | | | | VP1 | | | | |
| | Passage | 203 | 207 | 209 | 217 | 303 | 304 | 304 | 312 | 313 | 314 | 319 | 102 | 106 | 108 | 108 | 109 | 116 | 116 | 117 | 119 | 120 |
| | history* | 2 | 7 | 6 | 0 | 6 | 3 | 9 | 9 | 2 | 8 | 2 | 8 | 4 | 3 | 5 | 8 | 1 | 9 | 1 | 4 | 6/7 |
| KNP/19/89 | PK1RS1 | Iso | — | — | — | His | Thr | Gln | — | — | Pro | — | — | — | — | Gln | Arg | — | — | Thr | Phe | His- |
| | | | | | | | | | | | | | | | | | | | | | | Val |
| | PK1RS1BHK4 | Val | — | — | — | Asp | Ser | Glu | — | — | — | Thr | — | — | — | Arg | Thr | — | — | Ala | Leu | Asp- |
| | | | | | | | | | | | | | | | | | | | | | | Ala |
| ZIM/5/83 | BTY4RS1 | — | Met | — | — | — | — | — | — | — | — | — | Met | Ala | — | — | — | Glu | Tyr | — | — | — |
| ZIM/7/83 | BBHK4B1BHK5 | — | Thr | — | — | — | — | — | — | — | — | — | Val | Gly | — | — | — | Lys | His | — | — | — |
| ZAM/7/96 | | — | — | — | — | — | — | — | — | — | Glu | — | — | — | Glu | — | — | — | — | — | — | — |
| | | — | — | — | — | — | — | — | — | — | Lys | — | — | — | Lys | — | — | — | — | — | — | — |
| ZIM/14/90 | | — | — | Glu | Gln | — | — | — | Thr | Asp | — | — | — | — | — | — | — | — | — | — | — | — |
| | | — | — | Gln | Arg | — | — | — | Lys | Asn | — | — | — | — | — | — | — | — | — | — | — | — |

Evaluation of the recombinant vSAT1 revealed the accumulation of positively-charged residues Lys110 and Arg111 surrounding the five-fold axis of the virion, responsible for the acquisition of the ability to interact with HSPG receptors and replicate in CHO—K1 cells. An in-depth look at the residues present in this position of the SAR/9/81wt impala isolate (low passage) and BHK-21 adapted isolate (tc) showed that the cell culture adaptation of the SAT1 virus was accompanied by amino acid changes at positions 110 and 112 of the VP1 capsid protein. The 110NRG112 motif of the impala isolate, in this short βF-βG loop, changed to a mixture of Asn, His or Lys residues at location 110 and Arg, Lys or Asp at position 112 in the adapted strain. The amino acid variation correlated also with the mixed plaque phenotypes observed. The Arg111 in the 110NRG112 motif, in the absence of other positively-charged residues, was not sufficient for the acquisition of SAR/9/81wt to bind to HSPG and growth in CHO—K1 cells. The progeny viruses within the SAR/9/81tc population, on the other hand, were equipped with an altered surface-exposed positive patch neighboring the five-fold pore (FIG. 4), which provided the ability to utilize HSPG for cell entry in CHO—K1 and BHK-21 cells. This is consistent with the observed Lys residues at position 110 of vSAT1.

Similarly to SAR/9/81, the SAT1/KNP196/91^{P} isolate (P; wild-type isolated on primary pig kidney cells) revealed mainly large plaques with turbid edges on BHK-21 cells (FIG. 3). When the same isolate was adapted in BHK-21 cells (vac; vaccine strain), i.e. SAT1/KNP/196/91^{Vac}, it exhibited medium sized and small plaques with clear edges. CHO—K1 cells were susceptible to infection only with the latter isolate and plaques formed on CHO—K1 cell monolayers were of single small, clear-plaque phenotype. The SAT1/KNP196/91^{P} isolate did not form plaques on CHO—K1 cells. Genotypic changes during adaptation of SAT1/KNP196/91 virus included the same amino acids residues mapped for SAR/9/81 surrounding the pore at the 5-fold axis of the virion, i.e. the 1D residues 110 to 112 (Table 2; FIG. 4). The residues substitutions for KNP/196/91^{Vac} were KGR, compared to the KGG motif of the KNP196/91^{P} isolate. Additionally, a significant amino acid change was observed in the βB-βC loop of VP2 at position 74 (Table 2) where a Gln was substituted for an Arg in the vaccine strain. This latter residue is located on a surface exposed loop that surrounds the 3-fold axis of the virion (FIG. 4).

The SAT1/NAM/307/98 virus was previously isolated from buffalo (Syncerus caffer) in the West Caprivi Game Reserve, Namibia, in 1998 (Bastos et al., 2001; Storey et al., 2007). The primary isolate of this virus (SAT1/NAM/307/98^{P}) had difficulty to adapt to BHK-21 cells, and only after repeated cultivation in BHK-21 cells, it finally resulted in a variant (SAT1/NAM/307/98^{BHK}) revealing medium sized and small plaques with well-defined edges on BHK-21 cells. This variant was able to grow in CHO—K1 cells, as evident by the small plaques observed (FIG. 3). In contrast, NAM/307/98^{P} revealed turbid plaque morphology on BHK-21 cells that correlated with the slow replication rate observed for this virus in the same cells. Two amino acid substitutions of importance were Glu-Lys changes at positions 135 and 175 of VP3 (Table 2). Both changes mapped to surface exposed loops surrounding the 3-fold axes of the virion (FIG. 4). Adaptation of another SAT1 field isolate, ZAM/2/93, on BHK-21 cells was rapid (FIG. 3), with amino acid substitutions to positive charge residues at position 110-112 of VP1 (Table 2). The latter confirmed the domain 110-112 of VP1 as a hotspot to the accumulation of positive charges during cell culture adaptation.

Two vaccine strains belonging to the SAT2 serotype were investigated for disparate plaque morphologies during adaptation on BHK-21 to create vaccine master seed virus stocks (Table 1). The SAT2/KNP/19/89^{P} was isolated from buffalo in the Kruger National Park. This low passage isolate produced a mixture of medium to large sized plaques on BHK-21 cells with opaque edges, but CHO—K1 were unable to sustain growth of this isolate as observed by the absence of plaques. However, passaging four times on BHK-21 cells (SAT2/KNP/19/89^{Vac}) consequently revealed mostly medium-sized plaques with well-defined edges as well as growth on CHO—K1 cells (FIG. 3). The genetic differences between two related viruses, i.e. SAT2/ZIM/5/83 and SAT2/ZIM/7/83 were also studied. ZIM/7/83 is the vaccine derivative of ZIM/5/83 and the genetic changes were evidenced by the differences in plaque morphologies on BHK-21 cells. ZIM/5/83 produced mainly large plaques with opaque edges, similar to KNP/19/89^{P}, and its inability to replicate in CHO—K1 cells was indicative of the absence of a HSPG-binding phenotype. On the contrary, the high culture passage virus produced large clear plaques on BHK-21 cells and was able to infect and grow in CHO—K1 cells.

The amino acid substitutions in the SAT2 vaccine strains (Table 2), only Gln85→Arg and Glu161→Lys in VP1, showed significant charge difference on the surface of the virion (FIG. 4). The latter substitution follows two positively charged residues in VP1, i.e. Lys-His, at the C-terminal base of the G-H loop. The Gln85→Arg, found in KNP/19/89^{Vac}, is structurally surrounding the 5-fold axis of the virus and form part of a three amino acid domain in the βE-βF loop. This domain, HQR, in the primary pig kidney isolate changed to HRR in the vaccine strain.

Confirmation of the role of the three residue motif in VP1 at position 83-85 of SAT2 viruses came from the adaptation of the field isolate SAT2/ZAM/7/96 (Table 2), isolated from buffalo when the large plaques changed to a mixture of plaques and growth in CHO—K1 cells (FIG. 3). A Glu→Lys substitution was observed at position 83 of VP1 as well as residue 148 of VP3 (Table 2). Contrary to the abovementioned SAT2 viruses, the ability of high passage SAT2/ZIM/14/90 to infect and replicate CHO—K1 cells (FIG. 3) was associated with Gln170→Arg and Thr129→Lys substitutions in VP2 and VP3 respectively (Table 2).

In summary, the VP1 residues at position 110-112 of SAT1 viruses appear to be a “hotspot” to change during cell culture adaptation, while other distantly located residues in the capsid proteins may also be involved (74 of VP2, 135 and 175 of VP3). This site is unique to SAT1 viruses. Similarly, the residues 83-85 (noteworthy residue 86 is also a positive charge residue) of VP1 are prone to change during adaptation of SAT2 viruses.

Introduction of HSPG Binding Sites Into Chimeric Viruses

The application of the novel SAT HSPG-binding regions was investigated by introducing the positive charge amino acids into chimeric viruses that do not have this characteristic, using standard site-directed mutagenesis techniques. The two chimeric viruses chosen for this purpose included pNAM/SAT and pSAU/SAT, containing the outer capsid-coding region of the SAT1/NAM/307/98 and SAT2/SAU/6/00 cloned into the pSAT2 genetic backbone. The two chimeric viruses were selected for lacking the HSPG phenotype as measured by the inability to infect CHO—K1 cells. Neither of the two viruses was able to acquire this phenotype with repeated cultivation in BHK-21 cells. The putative HSPG-binding residues located adjacent to the 5-fold axes of the virion were introduced into the pNAM/SAT and pSAU/SAT. The most prominent and significant site observed for SAT1 viruses was the residues 110-112, where accumulation of positive charge residues was observed for three SAT1 isolates. The sequence of KRR was therefore introduced into the corresponding region of pNAM/SAT, which contained the sequence RGG. A site prone to accumulation of positive charge residues, during adaptation of SAT2 viruses in cell culture, was residues 1083 to 1085 of the VP1 protein. This motif surrounds the 5-fold axis of the virion. The KRK motif was located at the base of the GH-loop and was chosen as the second site to be introduced into pSAU/SAT.

The vNAM/SAT chimeric virus, containing the outer capsid proteins of the NAM/307/98 virus produced large, opaque plaques of BHK-21 cells, similar to the wild-type virus. The vNAMΔKRR mutant with the KRR motif introduced at residues 110-112 revealed plaque morphology similar to that of the recombinant vSAT1 virus. FIG. 5 shows that the plaques on BHK-21 cells were mainly small plaques with clearly defined edges. In addition, the vNAMΔKRR mutant was able to grow on CHO—K1 cells. This observation correlates with previous observations that adaptation is associated with a change in plaque phenotype and cell culture infectivity. This is the first report where adaptation phenotype, i.e. HSPG binding ability, was added to FMDV. The addition of the most significant putative SAT2 HSPG binding sites to the pSAU/SAT2 rendered mutant viruses that displayed a similar medium size, opaque plaque phenotype compared to the wild-type virus. The presence of a surface exposed negative charge Asp residue at position 83 that is in close proximity of the 85K residue mutation may result in a repulsive force on the negative charge sugar backbone of the cellular receptor and may prevent interaction with HSPG. Therefore, the residue D83 may need to be changed simultaneously.

A Full Potency Protection Experiment of a Chimera Vaccine in Pigs

An alternative approach in the development of inactivated vaccines involves the engineering of chimeric FMD viruses of which the antigenic properties can be readily manipulated. This recombinant DNA technology is unique in its application in FMD vaccines, as it allows for rapid alteration of the external capsid-coding region of a stable infectious clone to that of a current outbreak strain. In the present study, by engineering such a chimeric virus, a possible alternative to the conventional inactivated vaccine production of the SAT type viruses was investigated for the development of custom-engineered inactivated FMD vaccines. A cross-serotype chimeric virus, vKNP/SAT2, was constructed consisting of the external capsid-coding region of a SAT1 virus in the genetic backbone of a SAT2 virus. The viral progeny replicated stably in both FMD host and non-host species derived cell lines and the infective titres and ability to produce plaques were similar for the chimera and parental virus, from which it was derived. The efficient cell-entry ability of vKNP/SAT2 and high infectious particle production rates render chimeras that can be inactivated and utilised for vaccine manufacturing purposes.

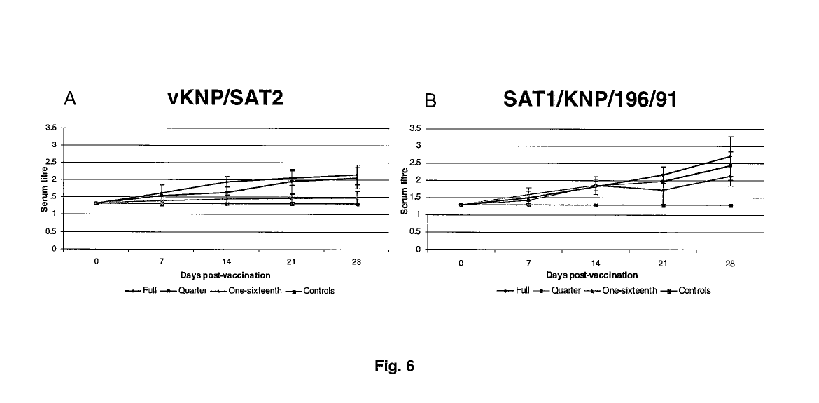

Two separate double oil emulsions incorporating inactivated 146S antigens of the chimera, vKNP/SAT2, and parental, KNP/196/91, were prepared and used for vaccination in a full potency trial (European Pharmacopoeia, 1997; OIE Manual of Standards, 2004). In order to monitor the antibody response elicited in pigs by the full (6.0 μg), quarter (1.5 μg) and one-sixteenth (0.375 μg) doses of the vaccines, sera samples collected were tested in a KNP/196/91-specific SPCE and the average titres for each vaccine dose were determined at weekly intervals (FIG. 6). Positive antibody titres were observed for most of the animals vaccinated with the three doses of both the chimera and parental vaccines. The full and quarter doses of the chimera vaccine elicited an antibody response comparable to the parental vaccine up to 21 days post-vaccination, whereafter the titres for the chimera remained similar until the day of challenge. In comparison, the parental vaccine elicited positive antibody responses for the full, quarter and one-sixteenth doses. Although the vKNP/SAT2 vaccine one-sixteenth dose did not induce a significant immune response, most animals were border-line positive at the time of challenge.

Serum neutralising antibody responses were measured by the VNT at the day of challenge for the vaccinated and control animals. All of the pigs were negative for FMDV-specific neutralising antibody at the onset of the study. At four weeks post-vaccination, 86.7% and 53.3% of the KNP/196/91 and vKNP/SAT2 vaccinated pigs were sero-positive on the VNT, respectively, especially those animals that received higher antigen doses. The chimeric vaccine induced high levels of homologous antibodies that cross reacted with the KNP/196/91 parental viruses; BHK-21 cell line-adapted and PK1RS4 isolates. Positive neutralising antibody titres were induced for the full doses of both vaccines. For the quarter dose of the chimera and parental vaccines, three and four animals, respectively, were positive for neutralisation of the KNP/196/91 virus. Whilst four pigs vaccinated with the parental one-sixteenth dose had positive neutralising antibody titres, the entire chimera one-sixteenth group was negative. Similar antibody response profiles were observed in both the SPCE and VNT for animals from all the groups and vaccinated with both antigens. Following challenge, none of the animals vaccinated with the KNP/196/91 vaccine developed lesions, while 60% of the animals receiving the chimeric vaccine were fully protected against disease. The onset of FMD lesions in animals with clinical disease was delayed and restricted in distribution, indicative of partial protection in these animals. By contrast, the onset of lesions in the control animals was faster than for those vaccinated with the chimera vaccine.

The vKNP/SAT2 displayed promising potential as a recombinant vaccine in its ability to retain phenotypic properties of the parental KNP/196/91 and the high titres achieved during infection resulted in high antigen yields that can readily be formulated as inactivated vaccine. In addition, the chimera and parental vaccines, elicited good humeral immune responses in pigs. The antibody titre increased more rapidly for the groups that received the higher antigen payloads of both vaccines. The onset of disease was delayed for the majority of the chimera vaccinated pigs when compared to the control animals and the clinical signs were less severe. Moreover, the majority of the pigs vaccinated with the chimera were protected against live virus challenge. This is indeed promising as the antigen range of up to 6 μg per dose is typically used in commercially available FMD vaccines.

Thus, more effective new generation inactivated vaccines for this highly infectious and economically important disease, which are custom-engineered and specifically produced for certain geographic localities, can be generated.

Methods

Cells, Viruses and Plasmids

Baby hamster kidney (BHK-21) cells, strain 21, clone 13 (ATCC CCL-10) were maintained as previously described (Rieder et al., 1993) and were used during transfection, virus recovery and plaque assays. Plaque assays were also performed using Chinese hamster ovary (CHO) cells strain K1 (ATCC CCL-61) maintained in Ham's F-12 medium (Invitrogen), supplemented with 10% FCS (Delta Bioproducts). Plaque assays were performed using a tragacanth overlay method and 1% methylene blue staining (Grubman et al., 1979; Rieder et al., 1993). Two SAT1 viruses i.e. a buffalo isolate, ZAM/2/93, and the vaccine strain, KNP/196/91; four SAT2 strains isolated from buffalo, i.e. ZIM/17/91, ZIM/14/90, ZIM/5/83 and ZAM/7/96, as well as the vaccine strain ZIM/7/83 and a SAT3 virus, KNP/19/89, utilized in vaccine manufacture were used in this study. Plasmids pSAT2, pNAM/SAT2 and pSAU/SAT2 have been described elsewhere (van Rensburg et al., 2004; Storey et al., 2007; Bohmer, MSc thesis 2004). The pSAT2 contains the genome-length cDNA of the wild-type FMDV SAT2 strain, ZIM/7/83, and was used in the construction of chimeric clones. Two SAT1 genome-length clones; constructed from a cell culture-adapted strain, (SAT1/SAR/9/81tc) and virus that was previously isolated from impala epithelium (SAR/9/81wt; wild-type), respectively, were also included for comparison.

Plasmid pSAT2 and derivatives of this plasmid have been described elsewhere (van Rensburg et al., 2004). The pSAT2 contains the genome-length cDNA of the wild-type FMDV SAT2 strain, ZIM/7/83, and unique SspI and XmaI cloning sites for the removal of the outer capsid and 2A-coding region.

RNA Extraction, cDNA Synthesis and Construction of Infectious Genome-Length cDNA and Chimeras

RNA was extracted from infected cell lysates using either a guanidium-based nucleic acid extraction method (Bastos, 1998) or TRizol® reagent (Life Technologies) according to the manufacturer's specifications and used as template for cDNA synthesis. Viral cDNA was synthesised with SuperScript III™ (Life Technologies) and oligonucleotide 2B208R (Knowles et al., 2009). The ca. 2.2 kb external capsid-coding regions of the viral isolates were obtained by PCR amplification with specific ologonucleotides to facilitate cloning or nucleotide sequence determination. Sequencing of the amplicons was performed using the ABI PRISM™ BigDye Terminator Cycle Sequencing Ready Reaction Kit v3.0 (Perkin Elmer Applied Biosystems).

pSAT1 plasmid carrying the genomic sequence of SAT1/SAR/9/81tc was constructed using a similar cloning strategy to the one employed by Rieder et al. (1993) and van Rensburg et al. (2004). The nucleotide sequence of the cloned regions was subsequently determined.

Construction of Mutant cDNA Clones

Site-directed mutagenesis of SAT capsid residues was carried out on plasmids pSAT1, pNAM/SAT2 and pSAU/SAT2 using an overlap extension mutagenesis method. The resulting ca. 2.2 kb DNA fragment was digested with SspI and XmaI (pNAM/SAT2 and pSAU/SAT2 mutations) and the external capsid-coding region (1B-1D/2A) was used to replace the corresponding region that was excised from the pSAT2. Alternatively, a ca. 3.2 kb fragment of the pSAT1 mutagenesis amplicon was digested with SnaB1 and Bln1 and cloned into the corresponding region of pSAT1. The mutations were verified by automated sequencing as described in section 2.2 and no second/other site mutations were found

In Vitro RNA Synthesis, Transfection and Virus Recovery

Plasmids containing genome-length cDNAs, chimeric cDNA or site-directed mutated cDNA clones were linearised at the SwaI site downstream of the poly-A tract and used as templates for RNA synthesis, using the MEGAscript™ T7 kit (Ambion). BHK-21 cell monolayers, in 35 mm diameter cell culture plates, were transfected with the in vitro-generated RNA using Lipofectamine2000™ (Life Technologies). Transfected monolayers were incubated at 37° C. with 5% CO₂ up to 48 hours in BME containing 1% FCS and 25 mM HEPES. The supernatants were used to infect BHK-21 monolayers and incubated for up to 48 hours at 37° C. Viruses were subsequently harvested by a freeze-thaw cycle and passaged four times in BHK-21 cells, using 10% of the supernatant of the previous passage, as described before (van Rensburg et al., 2004). Following the recovery of viable viruses the presence of the mutations were verified once more with automated sequencing.

Analysis of HSPG Utilization During Cell Entry of SAT Types of FMDV

The utilization of HSPG for cell entry was analyzed in CHO—K1 (positive for HSPG) cells which were infected with the specified viral strains and incubated for 1 hour and 24 hours, respectively, washed with MES-buffer (pH 4.0) to remove residual extracellular virus and frozen at −70° C. Virus titres were determined in BHK-21 cells and viral growth was calculated by subtracting the 24 hour titre results from the 1 hour titre results.

Amplification of High Passage Isolates in CHO—K1 Cells

BHK cell-adapted viruses were used to infect CHO—K1 cells for 1 hour, followed by an acidic wash step as described before, prior to incubation at 37° C. The viruses were harvested at greater than 90% CPE or at 48 hours and frozen at −80° C. The nucleotide sequences of the isolates with the ability to infect and produce greater than 90% CPE within 24 hours were determined and compared to those of the parental/original viruses.

Antigen Preparation and Vaccine Formulation

Following the original isolation (PK1RS4) from buffalo in the Kruger National Park, South Africa, the KNP/196/91 virus was passaged in cattle and BHK-21 cells (passage history: PK₁RS₄B₁BHK₄), prior to its application in engineering a genome-length construct by replacing the external capsid-coding region (1B-1D/2A) of the infectious cDNA clone SAT2/ZIM/7/83, pSAT2 (van Rensburg et al., 2004), with that of KNP/196/91 (pKNP/SAT2). The chimera, vKNP/SAT2, and parental, KNP/196/91, viruses harvested from infected BHK-21 monolayers were inactivated with 5 mM BEI for 26 h at 26° C., concentrated and purified as above. The genetic integrity of the viruses used for infection (passage 5) and vaccine formulation (passage 6) were verified. Two separate vaccine formulations, incorporating vKNP/SAT2 and KNP/196/91 inactivated 146S antigens as double oil emulsion (water-in-oil-in-water (WOW)) with Montanide ISA 206 (Seppic, Paris) were prepared. The appropriate antigen concentration was diluted in Tris-KCl buffer (0.1 M Tris, 0.3 M KCl, pH 7.5), followed by the addition of chloroform to a final concentration of 0.3% v/v. The oil adjuvant was mixed into the aqueous antigen phase (50:50) at 30° C. for 15 minutes and stored at 4° C. for 24 hours, followed by another brief mixing cycle for 5 minutes. A placebo vaccine was prepared for the control animals containing all the components except antigen.

Vaccination and Challenge of Pigs

Thirty-four, FMD-free female pigs, 3-4 months of age and weighing 25-30 kg were housed separately in six groups of five animals each (Groups 1-6) and one group of four controls (Group 7). Subsequent to an initial acclimation period, the pigs were vaccinated by the intramuscular route immediately caudal to the ear with 2 ml, 0.5 ml and 0.125 ml of 3 μg/ml of either vKNP/SAT2 (groups 1-3) or KNP/196/91 (groups 4-6) 146S antigen. The control group was administered a placebo formulation without antigen. Rectal temperatures and clinical signs were recorded daily. At 28 dpv the animals were inoculated intra-epidermally in the coronary band of the left hind heel bulb with 0.1 ml of 10⁵ TCID₅₀/ml challenge virus and examined daily for lesions, whereupon pigs were removed from the experiment. At day 10 post-infection the remainder of the animals were terminated. A body temperature equal to or greater than 39.6° C. was considered as mild fever, whereas temperatures equal to or greater than 40° C. were considered as severe fever. Serum samples were taken at 0, 7, 14, 21, 28 dpv and on the day of termination for serology.

Homologous challenge virus was prepared by three passages of KNP/196/91 (PK₁RS₄B₁BHK₄) in pigs. The pig adapted virus, designated PK₁RS₄B₁BHK₄P₃, was titrated in pigs, primary pig kidney (PK) cells and IB-RS-2 cells and the titre expressed as pig infective doses per ml (PID₅₀/ml) or tissue culture infective doses per ml (TCID₅₀/ml).

Pig Antibody Response Detected by Solid-Phase Competition ELISA

The presence of antibodies directed to SAT1 146S particles in sera was detected by a KNP/196/91-specific solid-phase competition ELISA (SPCE) that has been developed for this investigation. Trapping antibody and KNP/196/91 virus were added to the plates as above. Of each sample, 100 μl of an 1/20 dilution was added in triplicate and diluted 1:1 in 50 μl 0.5% (w/v) casein across the plate. Guinea pig anti-KNP/196/91 serum diluted 1/6000 in casein (50 μl) was added to the wells incubated and washed. The addition of antispecies conjugate and subsequent detection steps were as described before.

Specific Neutralising Antibody Against FMDV Detected by Virus Neutralisation Assay

Serum samples collected at 0 and 28 days post-vaccination were tested in the virus neutralisation test (VNT) for the presence of neutralising antibodies against FMDV. The VNT was carried out in micro-titre plates as described in the OIE Manual of Standards (2004). The serum samples were initially diluted 1/8, followed by a 1:1 dilution across the plate and the virus neutralising ability was tested against four dilutions of the homologous viruses (Esterhuysen et al., 1985). A regression line was calculated from the results and the 50% serum end-point titre at the log₁₀^{2.0}TCID₅₀ level established (Esterhuysen et al., 1988). Serum titres were expressed as the logarithm of the reciprocal of the final serum dilution to neutralise 100 TCID₅₀ of homologous FMDV in 50% of the wells, as calculated by the method of Kärber (1931).

While the invention has been described in detail with respect to specific embodiments thereof, it will be appreciated by those skilled in the art that various alterations, modifications and other changes may be made to the invention without departing from the spirit and scope of the present invention. It is therefore intended that the claims cover or encompass all such modifications, alterations and/or changes.

### REFERENCES

Almeida, M. R., Rieder, E., Chinsangaram, J., Ward, G., Beard, C., Grubman, M. J., Mason, P. W. 1998. Construction and evaluation of an attenuated vaccine for foot-and-mouth disease: difficulty adapting the leader proteinase-deleted strategy to the serotype O1 virus. Virus Res. 55, 49-60.

Bastos, A. D. S., Haydon, D. T., Forsberg, R., Knowles, N. J., Anderson, E. C., Bengis, R. G., Nel, L. H., Thomson, G. R. 2001. Genetic heterogeneity of SAT-1 type foot-and-mouth disease viruses in southern Africa. Arch. Virol. 146, 1537-1551.

Beard, C. W., Mason, P. W. 2000. Genetic determinants of altered virulence of Taiwanese foot-and-mouth disease virus. J. Virol. 74, 987-991.

Böhmer, MSc thesis 2004, Engineering of a chimeric SAT2 foot-and-mouth disease virus for vaccine production. Submitted in partial fulfilment of the requirements for the degree Master of Science in the Faculty of Natural and Agricultural Sciences Department of Microbiology and Plant Pathology University of Pretoria, South Africa

Esterhuysen, J. J., Thomson, G. R., Ashford, W. A., Lentz, D. W., Gainaru, M. D., Sayer, A. J., Meredith, C. D., Janse van Rensburg, D., Pini, A. 1988. The suitability of a rolled BHK₂₁ monolayer system for the production of vaccines against the SAT types of foot-and-mouth disease virus. I. Adaptation of virus isolates to the system, immunogen yields achieved and assessment of subtype cross reactivity. Onderstepoort J. Vet. Res. 55, 77-84.

Esterhuysen, J. J., Thomson, G. R., Flammand, J. R. B., Bengis, R. G. 1985. Buffalo in the northern Natal Game parks show no serological evidence of infection with foot-and-mouth disease virus. Onderstepoort J. Vet. Res. 52, 63-66.

European Pharmacopoeia, 5^{th} Ed. 1997. European Directorate for the Quality of Medicines, Strasbourg.

Fowler V. L., Paton D. J., Rieder E. and Barnett P. V. 2008. Chimeric foot-and-mouth disease viruses: evaluation of their efficacy as potential marker vaccines in cattle. Vaccine. 26(16):1982-9.

Grubman, M. J., Baxt, B., Bachrach, H. L. 1979. Foot-and-mouth disease virion RNA: studies on the relation between the lengths of its 3′-poly(A) segment and infectivity. Virol. 97, 22-31.

Jackson, T., Ellard, F. M., Abu-Ghazalch, R., Brooks, S. M., Blakemore, W. E., Corteyn, A. H., Stuart, D. L., Newman, J. W. I., King, A. M. Q. 1996. Efficient infection of cells in culture by type O foot-and-mouth disease virus requires binding to cell surface heparan sulfate. J. Virol. 70, 5282-5287.

Kärber, G. 1931. Beitrag zur kolletiven Behandlung pharmakologischer Reihenversuche. Archiv. Exp. Path. Pharm. 162, 480-483.

Cottam E M, Wadsworth J, Knowles N J, King D P. 2009. Full sequencing of viral genomes: practical strategies used for the amplification and characterization of foot-and-mouth disease virus. Methods Mol Biol., 551:217-30.

Sambrook, J. and Russel, D. W. Molecular Cloning: A laboratory Manual. Cold Spring Harbor Laboratory Press, Cold Spring Harbor, N.Y., 2001.

OIE manual of standards for diagnostic tests and vaccines. 5^{th} Ed 2004 Office International des Epizooties. Paris, France, 2004

Rieder, E., Bunch, T., Brown, F., Mason, P. W. 1993. Genetically engineered foot-and-mouth disease viruses with poly(C) tracts of two nucleotides are virulent in mice. J. Virol. 67, 5139-5145.

Sa-Carvalho, D., Rieder, E., Baxt, B., Rodarte, R., Tanuri, A., Mason, P. W. 1997. Tissue culture adaptation of foot-and-mouth disease virus selects viruses that bind to heparin and are attenuated in cattle. J. Virol. 71, 5115-5123.

Storey P., Theron J., Maree F. F., O'Neill H. G. 2007. A second RGD motif in the 1D capsid protein of a SAT1 type foot-and-mouth disease virus field isolate is not essential for attachment to target cells. Virus Res. 124(1-2), 184-92.

Van Rensburg, H. G., Henry, T. M., Mason, P. W. 2004. Studies of genetically defined chimeras of a European type A virus and a South African Territories type 2 virus reveal growth determinants for the foot-and-mouth disease virus. J. Gen. Virol. 85, 61-68.

Zhao, Q., Pacheco, J. M., Mason, P. W. 2003. Evaluation of genetically engineered derivatives of a Chinese strain of foot-and-mouth disease virus reveals a novel cell-binding site which functions in cell culture and in animals. J. Virol. 77, 3269-3280.

Zibert, A., Maass, G., Strebel, K., Falk, M. M., Beck, E. 1990. Infectious foot-and-mouth disease virus derived from a cloned full-length cDNA. J. Virol. 64, 2467-2473.

What is claimed is:

1. A chimeric foot and mouth disease virus (FMDV) nucleic acid molecule encoding a first FMDV strain, wherein nucleotides encoding an outer capsid region have been replaced with nucleotides encoding an outer capsid region of a second FMDV strain which includes or has been modified to introduce a heparan sulphate proteoglycan binding site.

2. The nucleic acid molecule of claim 1, wherein the first FMDV strain is selected from the group consisting of SAT1, SAT2, SAT3, A, C, O and Asia 1 serotypes.

3. The nucleic acid molecule of claim 1, wherein the second FMDV strain is selected from the group consisting of SAT1, SAT2, SAT3, A, C, O and Asia 1 serotypes.

4. The nucleic acid molecule of claim 1, wherein the first and second FMDV strains are different serotypes.

5. The nucleic acid molecule of claim 1, wherein the first FMDV strain is a strain which is able to grow in vitro on a commercial scale.

6. The nucleic acid molecule of claim 1, wherein the second strain is a wild-type strain in current circulation.

7. The nucleic acid molecule of claim 1, wherein the heparan sulphate proteoglycan binding site is introduced by modifying one or more nucleotides of the outer capsid region of the second FMDV strain to encode:
(a) lysine or arginine at residue 110 of SAT1 VP1 (SEQ ID NO: 22);
(b) lysine or arginine at residue 112 of SAT1 VP1 (SEQ ID NO: 22);
(c) lysine or arginine at residue 135 of SAT1 VP3 (SEQ ID NO: 24);
(d) lysine or arginine at residue 175 of SAT1 VP3 (SEQ ID NO: 24);
(e) lysine or arginine at residue 74 of SAT1 VP2 (SEQ ID NO: 23);
(f) lysine or arginine at residue 83 of SAT2 VP1 (SEQ ID NO: 25);
(g) lysine or arginine at residue 85 of SAT2 VP1 (SEQ ID NO: 25);
(h) lysine or arginine at residue 161 of SAT2 VP1 (SEQ ID NO: 25); or
(i) lysine or arginine at an equivalent position of one or more of (a)-(h) of another strain.

8. The nucleic acid molecule of claim 7, wherein nucleotides encoding amino acid residues at positions 110 and 112 of VP1 (SEQ ID NO: 22) or at positions 135 and 175 of VP3 (SEQ ID NO: 24) are additionally modified to encode a lysine or arginine residue if the second FMDV is a SAT1 serotype.

9. The nucleic acid molecule of claim 7, wherein nucleotides encoding amino acid residues at positions 83 and 85 of VP1 or at position 161 of VP1 (SEQ ID NO: 25) are additionally modified to encode a lysine or arginine residue if the second FMDV is a SAT2 serotype.

10. The nucleic acid molecule of claim 1, wherein the first FMDV strain has at least 70% sequence identity to SEQ ID NO: 1 or SEQ ID NO: 2, or an RNA equivalent thereof.

11. The nucleic acid molecule of claim 1, wherein the capsid encoding region of the second FMDV strain is a DNA or RNA sequence encoding the amino acid sequence of SEQ ID NOs: 3, 4 or 5, or a sequence which has at least 70% sequence identity thereto.

12. A vector comprising a nucleic acid molecule of claim 1.

13. A host cell comprising a nucleic acid molecule of claim 1.

14. The host cell of claim 13, which is a BHK-21 cell.

15. A virus comprising the nucleic acid molecule of claim 1.

16. The virus of claim 15, which is inactivated.

17. A composition comprising the virus of claim 15 and an adjuvant.

18. The composition of claim 17, wherein the virus is inactivated.

19. The composition of claim 17 for use in eliciting an immune response against FMDV in a subject.

20. A method of eliciting an immune response to FMDV in a subject, comprising administering the virus of claim 16 or the composition of claim 18 to the subject.

21. A method of producing a chimeric FMDV nucleic acid molecule, the method comprising the steps of:
modifying a nucleotide sequence encoding an external capsid protein of a first FMDV strain to include a heparan sulphate proteoglycan (HSPG) binding site; and
inserting the modified capsid-coding nucleotide sequence into a nucleotide sequence of a second FMDV strain.

22. The method according to claim 21, wherein the modified capsid-coding nucleotide sequence of the first FMDV strain replaces nucleotides encoding the external capsid protein of the second FMDV strain.

23. The method of claim 21, wherein another nucleotide sequence encoding another capsid protein of the first FMDV strain is additionally inserted into the second FMDV strain.

24. The method of claim 21, wherein the first and second FMDV strains are the same or different serotypes and are selected from serotypes SAT1, SAT2, SAT3, A, C, O and Asia 1.

25. The method of claim 21, wherein the heparan sulphate proteoglycan binding site is introduced by modifying one or more nucleotides of the outer capsid region of the second FMDV strain to encode:
(a) lysine or arginine at residue 110 of SAT1 VP1 (SEQ ID NO: 22);
(b) lysine or arginine at residue 112 of SAT1 VP1 (SEQ ID NO: 22);
(c) lysine or arginine at residue 135 of SAT1 VP3 (SEQ ID NO: 24);
(d) lysine or arginine at residue 175 of SAT1 VP3 (SEQ ID NO: 24);
(e) lysine or arginine at residue 74 of SAT1 VP2 (SEQ ID NO: 23);
(f) lysine or arginine at residue 83 of SAT2 VP1 (SEQ ID NO: 25);
(g) lysine or arginine at residue 85 of SAT2 VP1 (SEQ ID NO: 25);
(h) lysine or arginine at residue 161 of SAT2 VP1 (SEQ ID NO 25); or
(i) lysine or arginine at an equivalent position of one or more of (a)-(h) of another serotype.

26. The method of claim 25, wherein nucleotides encoding amino acid residues at positions 110 and 112 of VP1 (SEQ ID NO: 22) or at positions 135 and 175 of VP3 (SEQ ID NO: 24) are additionally modified to encode a lysine or arginine residue if the first FMDV strain is a SAT1 serotype.

27. The method of claim 25, wherein nucleotides encoding amino acid residues at positions 83 and 85 of VP1 or at position 161 of VP1 (SEQ ID NO: 25) are additionally modified to encode a lysine or arginine residue if the first FMDV strain is a SAT2 serotype.
